(19) United States
(12) Patent Application Publication
(10) Pub. No.: US 2023/0348858 A1
(43) Pub. Date: Nov. 2, 2023

(54) COMPOSITIONS AND METHODS FOR PROMOTING PROLIFERATION IN CARDIOMYOCYTES

(71) Applicant: THE TRUSTEES OF INDIANA UNIVERSITY, Bloomington, IN (US)

(72) Inventors: Ching-Pin CHANG, San Mateo, CA (US); Jin YANG, Carmel, IN (US); Xuhui FENG, Indianapolis, IN (US)

(21) Appl. No.: 17/923,055

(22) PCT Filed: May 8, 2020
(86) PCT No.: PCT/US2020/032192

§ 371 Date: Nov. 3, 2022

(51) Int. Cl. C12N 5/077; C12N 9/02

(52) U.S. Cl. CPC C12N 5/0657; C12N 9/0071; C12N 2501/71; C12N 2501/155

(57) ABSTRACT

Disclosed are methods and compositions for inducing proliferation in cardiomyocyte cells or for high-throughput assays.

### STATEMENT OF GOVERNMENT SUPPORT

This invention was made with government support under HL118087 and HL121197 awarded by the National Institutes of Health. The Government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 31 kilobytes ACII (Text) file named “317699_ST25.txt,” created on May 8, 2020.

BACKGROUND OF THE DISCLOSURE

Recent studies began to elucidate mechanisms that underlie the regulation of N⁶-methyladenosine (m⁶A), a covalent modification of polyadenylated RNAs to control gene expression. The physiological impacts of m⁶A regulation are largely unknown and have raised intense interests in the research community. Only a few proteins are known to write, read, or erase m⁶A marks on RNAs. The writers METTL3 and METTL14 form an m⁶A methyltransferase complex with WTAP to deposit m⁶A marks on RNAs, whereas the readers YTH domain-containing proteins recognize m⁶A and target m⁶A-marked RNAs to P-bodies for accelerated degradation. Both m⁶A writers and readers have multiple members that are highly conserved in most eukaryotes, including yeast, flies, and mammals. In contrast to those m⁶A writers and readers, only two proteins (FTO and ALKBH5) are known to erase RNA m⁶A marks, and these two erasers have no yeast homologues. Therefore, it remains an issue of debate whether m⁶A erasers exist in lower eukaryotes. In yeast, although the m⁶A methyltransferase complex Ime4 is crucial for meiosis, no yeast version of m⁶A eraser has been identified, and this represents a major gap in eukaryotic m⁶A regulatory loop.

### SUMMARY

This disclosure provides useful compositions and methods based on the inventor's recently discovered mechanism of action. The present disclosure relates generally to the methods of promoting proliferation in cardiomyocyte cells. More specifically, the present disclosure relates to the use of an engineered protein to demethylate Bmp 10 mRNA. In one embodiment the engineered protein comprises a sequence having at least 95% sequence identity to SEQ ID NO 2. A representative listing of relevant sequence identifiers is as follows:
SEQ ID NO 1 is an amino acid sequence of Brg1;
SEQ ID NO 2 is an amino acid sequence of D1D2 motif encompassing D1-Insertion-D2 of Brg1;
SEQ ID NO 3 is an amino acid sequence of R/R+H/D Motif of D1D2;
SEQ ID NO 4 is R/R motif of Brg1;
SEQ ID NO 5 is H/D motif of Brg1;
SEQ ID NO 6 is an amino acid sequence of D1 motif of Brg1;
SEQ ID NO 7 is an amino acid sequence of D2 motif of Brg1;
SEQ ID NO 8 is a DNA sequence of Bmp10;
SEQ ID NO 9 is an mRNA sequence of Region 2 of Bmp10;
SEQ ID NO 10 is an mRNA sequence of site M2+M3 of Region 2;
SEQ ID NO 11 is an mRNA sequence of site M2 of Region 2;
SEQ ID NO 12 is an engineered mRNA sequence with a methylated adenosine; and
SEQ ID NO 27 is an amino acid sequence for MBP.

In one embodiment, a method of promoting cardiomyocyte cell proliferation comprises providing a demethylase to the cardiomyocyte, wherein the demethylase is designed to remove a methyl group from Region 2 of the Bmp10 mRNA.

In one embodiment, a method of inducing proliferation in a cardiomyocyte cell comprises providing an m⁶A methylation inhibitor to the cardiomyocyte cell.

In one embodiment, a method of inducing proliferation in a cardiomyocyte cell comprises providing an engineered protein comprising a demethylase to the cell, wherein the demethylase enhances the Brg1 demethylation activity, wherein the engineered protein comprises an amino acid sequence having at least 95% sequence identity to SEQ ID NO 2, wherein the Brg1 demethylation activity is enhanced relative to the base line level of Brg1 in the cardiomyocyte cell.

In one embodiment, a method of testing lead compounds in a high-throughput assay comprises providing a recombinant protein comprising an amino acid sequence having at least 95% sequence identity to SEQ ID NO 2; providing a recombinant mRNA comprising a nucleic acid sequence and a methylated adenosine; providing a lead compound; and analyzing the effect of the lead compound on the recombinant protein's ability to demethylate the methylated adenosine of the recombinant mRNA.

In one embodiment, a method of enhancing proliferation of cardiomyocytes, comprises contacting the cardiomyocytes with an effective amount of a pharmaceutical composition to remove a methyl group from Region 2 of Bmp10 mRNA, wherein the pharmaceutical composition includes a demethylase and a pharmaceutically acceptable carrier.

In one embodiment, a method of culturing cardiomyocyte cells, comprises introducing a pharmaceutical composition into a cardiomyocyte culture medium, wherein the pharmaceutical composition includes a demethylase and pharmaceutically acceptable carrier.

In one embodiment, a culture medium comprises a buffer, and a pharmaceutical composition that includes a demethylase and a pharmaceutically acceptable carrier, wherein the amount of demethylase present in the culture medium is effective to remove a methyl group from Region 2 of Bmp10 mRNA when in contact with a cardiomyocyte.

In one embodiment, a method of producing a culture medium, comprises mixing a buffer with a demethylase, wherein the amount of demethylase present in the buffer is effective to remove a methyl group from Region 2 of Bmp10 mRNA when the demethylase is in contact with a cardiomyocyte.

In one embodiment, an engineered protein comprises an amino acid sequence having at least 95% sequence identity to SEQ ID NO 2.

### BRIEF DESCRIPTION OF THE DRAWINGS

FIG. 1 is an illustration depicting the D1D2 motif showing the D1, Insertion, and D2 Domains of Brg1 and showing where the R/R motif is located by a star and the H/D motif by the hexagon, and further showing sequence alignments of R/R and H/D motifs in yeast, drosophila, zebrafish, mouse, and human.

FIG. 2 is a graph representing the quantitation of captured methylated adenosine (m⁶A) in samples from control hearts and hearts where Brg1 was knocked out, and showing a dot blot above the graph indicating, along with the graph, that more m⁶A was captured.

FIG. 3 is a graph representing the quantitation of the relative m⁶A pulled down comparing a control heart to a knockout Brg1 E10.5 mouse heart and comparing across four regions of Bmp10 mRNA showing that R2 (“Region 2”) showed an increase in captured methylated adenosine compared to the other three regions. Additionally, FIG. 3 shows a map of the four regions analyzed on an illustrative depiction of Bmp10 mRNA.

FIG. 4 is a graph representing the quantitation of m⁶A RNA-immunoprecipitation (RNA-IP) of Bmp10 Region 2 in SW13 cells transfected with Bmp10 expression plasmids that contain different M1 through M6 mutations.

FIG. 5 is a graph representing a miCLIP of E 10.5 control and Brg1 knockout mouse hearts and showing mutations occurring at positions 0 and +2 of site M2 of Region 2 on Bmp10 mRNA.

FIG. 6 is a graph representing a MazF Digestion and the methylation increased at site M2+M3 (as shown in FIG. 21), for knockout Brg1 E10.5 mouse heart compared to control E10.5 mouse hearts.

FIG. 7 is a graph showing that Ythdf2 bound to methylated Bmp10 mRNA in the knockout mouse because Brg1 was not present to remove the methyl group in Region 2 compared to the control embryonic hearts which had a Brg1 protein present.

FIG. 8 is a graph showing RNA-IP of YTHDF2-Bmp10 in SW13 cells co-transfected with Brg1 and Bmp10 expression plasmids.

FIG. 9 is a graph showing RNA-IP of YTHDF2-Bmp10 in SW13 cells transfected with expression plasmids of wildtype Bmp10 (WT) or Bmp10 with a site M2 mutation (M2).

FIG. 10 is a graph showing RT-qPCR analysis of Bmp10 expression in E10.5 control and Brg1 knockout hearts with or without 3-DZA treatment.

FIG. 11 is a graph representing the quantification of RNA-IP of Bmp10 at R2 regions in E10.5 control and knockout hearts with or without 3-DZA treatment.

FIG. 12 is a graph representing the quantification of ventricle thickness of E10.5 control and knockout hearts with or without 3-DZA treatment.

FIG. 13 is a graph representing the quantification of BrdU staining of myocardium in E10.5 control and Brg1 knockout hearts with or without treatment.

FIG. 14 is a graph representing an m⁶A dot blot quantification in BRG1 knockdown SW13 cells transfected with BRG1 or BRG1ΔD1D2.

FIG. 15 is a graph representing m⁶A dot blot quantification activities of MBP, MBP-D1D2 recombinant protein, and full length Brg1 protein's ability to demethylate the Bmp10 mRNA sequence with methylated adenosine. MBP=Maltose Binding Protein.

FIG. 16 is a graph representing m⁶A dot blot quantification activities of MBP, MBP-D1D2, MBP-D1, and MBP-D2 recombinant proteins' ability to demethylate the Bmp10 mRNA sequence with methylated adenosine.

FIG. 17 is a graph representing the enzyme kinetics of recombinant protein D1D2 catalyzed demethylation.

FIG. 18 is a graph and dot blot analysis representing the m⁶A demethylating activities of MBP, MBP-D1D2, MBP-D1D2R973Q, and MBP-D1D2H1173A/D1175A recombinant proteins.

FIG. 19 is an illustration of Brg1's role in demethylating Bmp10 mRNA in a cardiomyocyte.

FIG. 20 is a D1D2 sequence alignment and motif analysis in different species specifically showing the D1D2 motifs and within those motif's the R/R motif and the H/D motif within various species.

FIG. 21 is an illustration of six sites M1 through M6, and showing mutation sites tried at each of those m⁶A sites in Region 2 of Bmp10 DNA.

FIG. 22 is a schematic diagram of miCLIP process and the difference in enzyme cutting when site M2 is methylated.

FIG. 23 is an m⁶A dot blot analysis of demethylating activities of MBP, MBP-D1D2 engineered protein, and full length Brg1 protein on generic RNA probe containing m⁶A site.

FIG. 24 is an m⁶A dot blot analysis of demethylating activities of MBP, MBP-D1 engineered protein, MBP-D2 engineered protein, and MBP-D1D2 engineered protein on generic RNA probe containing m⁶A site.

### DETAILED DESCRIPTION

### Definitions

In describing and claiming the invention, the following terminology will be used in accordance with the definitions set forth below.

As used herein, the term “Brg1” refers to Brahma-related gene-1, a protein generally understood to be involved in numerous chromatin-modifying enzymatic complexes, and in this disclosure was discovered to have a separate mechanism of action as a demethylase in cardiomyocytes as well as other cell types.

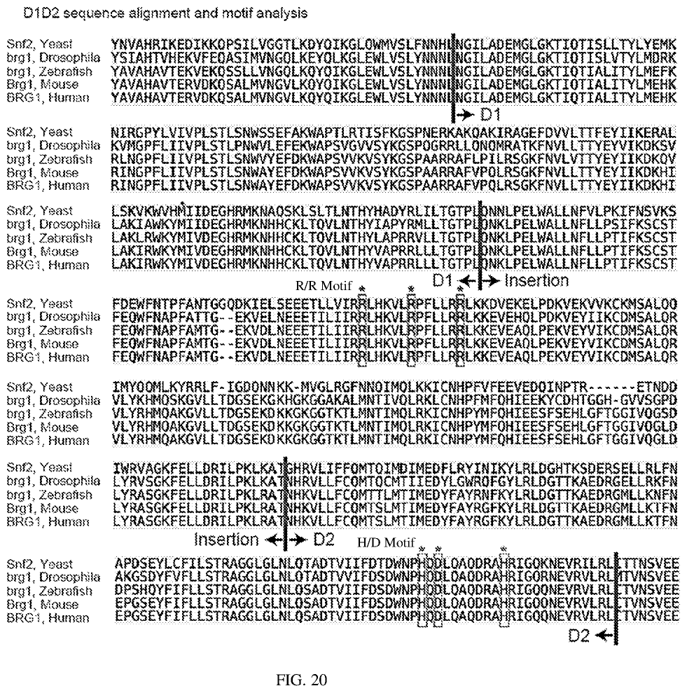

As used herein, the term “D1D2” refers to Brg1's helicase domain comprising D1 coupled to Insertion coupled to D2 as illustrated in FIG. 1 and FIG. 20. It is shown here that within D1D2 are the amino acids responsible for the catalytic function of removing methyl groups from adenosine.

As used herein, the term “R/R+H/D” refers to specific amino acid sequences found within the Insertion and D2 regions of the Brg1 helicase domain, as shown in FIG. 1 and FIG. 20, wherein the R/R motif has an amino acid sequence of IRRLHKVLRPFLLRRLK, and the H/D motif has an amino acid sequence of DWNPHQDLQAQDRAHRI, wherein the underlined amino acids show some amino acids of interest.

As used herein, the term “Bmp10” mRNA refers to bone morphogenetic protein-10 mRNA.

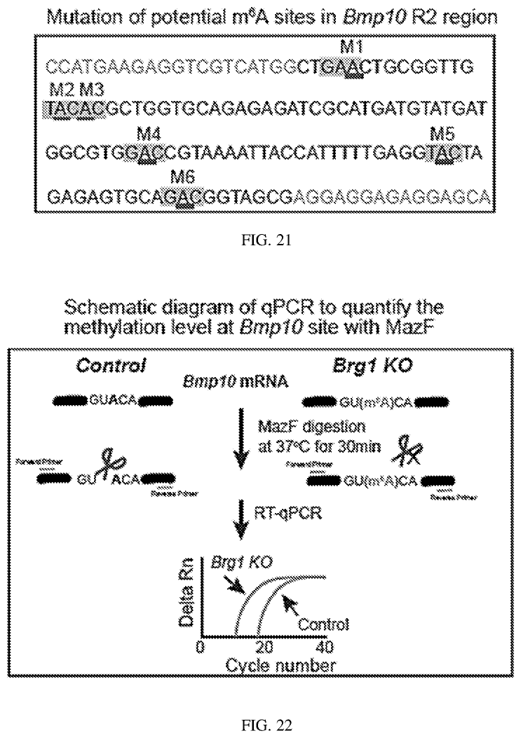

As used herein, the term “Region 2” refers to a section of Bmp10 mRNA where D1D2 interacts and removes the m⁶A from Bmp10 mRNA, and is illustrated in FIG. 3 and FIG. 21. Region 2 has a nucleic acid sequence of:

CCAUGAAGAGGUCGUCAUGGCUGA[A]CUGCGGUUGU[A]C[A]CGCUGGUGCAGAG AGAUCGCAUGAUGUAUGAUGGCGUGG[A]CCGUAAAAUUACCAUUUUUGAGGU[A]CUAGAGAGUGCAG[A]CGGUAGCGAGGAGGAGAGGAGCA; wherein the underlined nucleic acids identify sites M1, M4, M5, and M6, respectively, and the double underlined nucleic acids identify site M2+M3. The [A] represents the position of a possible methylated adenosine.

As used herein, the term “site M2+M3” refers to represents amino acids GU[A]C[A]C, wherein the [A] represents a site for a methylated adenosine, within Region 2 of the Bmp10 mRNA.

As used herein, the term “Site M2” refers to GU[A] within Region 2.

As used herein, the term “lead compound” refers to a compound of commercial or research interest by an institution or organization.

As used herein, the term “engineered” includes the term recombinant, and refers to protein or mRNA not naturally found in the body because it includes some kind of modification including, but not limited to, a truncation of the wildtype amino acid or nucleic acid sequence, a mutation, or the addition of a probe. In some embodiments, engineered also encompasses a sequence, mRNA, or protein that is substantially purified.

### Embodiments

This disclosure provides methods and compositions based on a newly discovered mechanism of Brg1. Brg1, a protein, is understood to play a role in chromatin remodeling and organization. Here, the methods and compositions take advantage of a newly discovered non-chromatin remodeling mechanism of Brg1. This disclosure provides methods and compositions based on the newly discovered demethylase activity of Brg1.

In one embodiment, an engineered protein is provided. The engineered protein comprises an amino acid sequence having at least 95% sequence identity to SEQ ID NO 2. In one embodiment, the engineered protein comprises an amino acid sequence having at least 95% sequence identity to SEQ ID NO 3. In one embodiment, the engineered protein comprises an amino acid sequence having at least 95% sequence identity to SEQ ID NO 1. The engineered protein may be used to induce or enhance cardiomyocyte cell proliferation for various uses including cell therapies or for the development of high-throughput screening assays.

In one embodiment, the engineered protein comprises an amino acid sequence having 95% sequence identity to SEQ ID NO 1. In one embodiment, the engineered protein comprises an amino acid sequence having at least 90%, 91%, 92%, 93%, 94%, 95%, 96%, 97%, 98%, or 99% sequence identity to SEQ ID NO 1. In one embodiment, the engineered protein further comprises a probe. The probe may be an antibody, a protein, a bead, a fluorophore, a radioisotope, or a combination thereof. In one embodiment, the engineered protein consists essentially of a probe coupled to an amino acid sequence having at least 95% sequence identity to SEQ ID NO 1. In one embodiment, the engineered protein consists essentially of a probe coupled to an amino acid sequence having at least 95%, 96%, 97%, 98%, or 99% sequence identity to SEQ ID NO 1. In one embodiment, the engineered protein consists essentially of a probe coupled to an amino acid sequence of SEQ ID NO 1. In one embodiment, the engineered protein comprises a biotin coupled to an amino acid sequence having at least 95%, 96%, 97%, 98%, or 99% sequence identity to SEQ ID NO 1. In one embodiment, an engineered protein comprises a biotin coupled to SEQ ID NO 1. In one embodiment, an engineered protein consists essentially of a biotin coupled to SEQ ID NO 1. In one embodiment, an engineered protein comprises an amino acid sequence of SEQ ID NO 27 coupled to SEQ ID NO 1.

In one embodiment, the engineered protein comprises an amino acid sequence having at least 90%, 91%, 92%, 93%, 94%, 95%, 96%, 97%, 98%, or 99% sequence identity to SEQ ID NO 2. In one embodiment, an engineered protein comprises a probe and an amino acid sequence having at least 95%, 96%, 97%, 98%, or 99% sequence identity to SEQ ID NO 2. In one embodiment, an engineered protein consists essentially of a probe and an amino acid sequence having at least 95%, 96%, 97%, 98%, or 99% sequence identity to SEQ ID NO 2. In one embodiment, the engineered protein consists essentially of a probe coupled to an amino acid sequence of SEQ ID NO 2. In one embodiment, the engineered protein comprises a biotin coupled to an amino acid sequence having at least 95%, 96%, 97%, 98%, or 99% sequence identity to SEQ ID NO 2. In one embodiment, an engineered protein comprises a biotin coupled to SEQ ID NO 2. In one embodiment, an engineered protein consists essentially of a biotin coupled to SEQ ID NO 2. In one embodiment, an engineered protein comprises an amino acid sequence of SEQ ID NO 27 coupled to SEQ ID NO 2.

In one embodiment, the engineered protein comprises an amino acid sequence having at least 90%, 91%, 92%, 93%, 94%, 95%, 96%, 97%, 98%, or 99% sequence identity to SEQ ID NO 3. In one embodiment, an engineered protein comprises a probe and an amino acid sequence having at least 95%, 96%, 97%, 98%, or 99% sequence identity to SEQ ID NO 3. In one embodiment, an engineered protein consists essentially of a probe and an amino acid sequence having at least 95%, 96%, 97%, 98%, or 99% sequence identity to SEQ ID NO 3. In one embodiment, the engineered protein consists essentially of a probe coupled to an amino acid sequence of SEQ ID NO 3. In one embodiment, the engineered protein comprises a biotin coupled to an amino acid sequence having at least 95%, 96%, 97%, 98%, or 99% sequence identity to SEQ ID NO 3. In one embodiment, an engineered protein comprises a biotin coupled to SEQ ID NO 3. In one embodiment, an engineered protein consists essentially of a biotin coupled to SEQ ID NO 3. In one embodiment, an engineered protein comprises an amino acid sequence of SEQ ID NO 27 coupled to SEQ ID NO 3.

In one embodiment, the engineered protein comprises an amino acid sequence having at least 90%, 91%, 92%, 93%, 94%, 95%, 96%, 97%, 98%, or 99% sequence identity to SEQ ID NO 4. In one embodiment, an engineered protein comprises a probe and an amino acid sequence having at least 95%, 96%, 97%, 98%, or 99% sequence identity to SEQ ID NO 4. In one embodiment, an engineered protein consists essentially of a probe and an amino acid sequence having at least 95%, 96%, 97%, 98%, or 99% sequence identity to SEQ ID NO 4. In one embodiment, the engineered protein consists essentially of a probe coupled to an amino acid sequence of SEQ ID NO 4. In one embodiment, the engineered protein comprises a biotin coupled to an amino acid sequence having at least 95%, 96%, 97%, 98%, or 99% sequence identity to SEQ ID NO 4. In one embodiment, an engineered protein comprises a biotin coupled to SEQ ID NO 4. In one embodiment, an engineered protein consists essentially of a biotin coupled to SEQ ID NO 4. In one embodiment, an engineered protein comprises an amino acid sequence of SEQ ID NO 27 coupled to SEQ ID NO 4.

In one embodiment, the engineered protein comprises an amino acid sequence having at least 90%, 91%, 92%, 93%, 94%, 95%, 96%, 97%, 98%, or 99% sequence identity to SEQ ID NO 5. In one embodiment, an engineered protein comprises a probe and an amino acid sequence having at least 95%, 96%, 97%, 98%, or 99% sequence identity to SEQ ID NO 5. In one embodiment, an engineered protein consists essentially of a probe and an amino acid sequence having at least 95%, 96%, 97%, 98%, or 99% sequence identity to SEQ ID NO 5. In one embodiment, the engineered protein consists essentially of a probe coupled to an amino acid sequence of SEQ ID NO 5. In one embodiment, the engineered protein comprises a biotin coupled to an amino acid sequence having at least 95%, 96%, 97%, 98%, or 99% sequence identity to SEQ ID NO 5. In one embodiment, an engineered protein comprises a biotin coupled to SEQ ID NO 5. In one embodiment, an engineered protein consists essentially of a biotin coupled to SEQ ID NO 5. In one embodiment, an engineered protein comprises an amino acid sequence of SEQ ID NO 27 coupled to SEQ ID NO 5.

In one embodiment, a high-throughput method of testing lead compounds is provided. In one embodiment, the method comprises providing an engineered protein, a methylated mRNA, and a lead compound. The method further includes the step of analyzing the effect of the lead compound on the engineered protein's ability to remove the methyl group from the mRNA.

In one embodiment, a high-throughput method of testing lead compounds comprises providing an engineered protein, a methylated mRNA, and a lead compound. In one embodiment, the engineered protein comprises an amino acid sequence having at least 95%, 96%, 97%, 98%, or 99% sequence identity to SEQ ID NO 2. In one embodiment, the engineered protein further comprises a probe coupled to the engineered protein. In one embodiment, the engineered protein consists essentially of a probe coupled to an amino acid sequence having at least 95%, 96%, 97%, 98%, or 99% sequence identity to SEQ ID NO 2. In one embodiment, the engineered protein consists essentially of an amino acid sequence having at least 95%, 96%, 97%, 98%, or 99% sequence identity to SEQ ID NO 2.

In one embodiment, a high-throughput method of testing lead compounds comprises providing an engineered protein, a methylated mRNA, and a lead compound. In one embodiment, the engineered protein comprises an amino acid sequence having at least 95%, 96%, 97%, 98%, or 99% sequence identity to SEQ ID NO 3. In one embodiment, the engineered protein further comprises a probe coupled to the engineered protein. In one embodiment, the engineered protein consists essentially of a probe coupled to an amino acid sequence having at least 95%, 96%, 97%, 98%, or 99% sequence identity to SEQ ID NO 3. In one embodiment, the engineered protein consists essentially of an amino acid sequence having at least 95%, 96%, 97%, 98%, or 99% sequence identity to SEQ ID NO 3. In another embodiment, the engineered protein comprises an amino acid sequence having at least 95%, 96%, 97%, 98%, or 99% sequence identity to SEQ ID NO 3. In one embodiment, the engineered protein further comprises a probe. In another embodiment, the engineered protein comprises an amino acid sequence having at least 95%, 96%, 97%, 98%, or 99% sequence identity to SEQ ID NO 3. In one embodiment, the engineered protein further comprises a probe.

In one embodiment, a high-throughput method of testing lead compounds comprises providing an engineered protein, a methylated mRNA, and a lead compound. In one embodiment, the engineered protein comprises an amino acid sequence having at least 95%, 96%, 97%, 98%, or 99% sequence identity to SEQ ID NO 1. In one embodiment, the engineered protein further comprises a probe coupled to the engineered protein. In one embodiment, the engineered protein consists essentially of a probe coupled to an amino acid sequence having at least 95%, 96%, 97%, 98%, or 99% sequence identity to SEQ ID NO 1. In one embodiment, the engineered protein consists essentially of an amino acid sequence having at least 95%, 96%, 97%, 98%, or 99% sequence identity to SEQ ID NO 1.

In one embodiment, a high-throughput method of testing lead compounds comprises providing an engineered protein, a methylated mRNA, and a lead compound. In one embodiment, the methylated mRNA comprises a nucleic acid sequence having at least 95%, 96%, 97%, 98%, or 99% sequence identity to SEQ ID NO 8. In one embodiment, the methylated mRNA further comprises a probe, wherein the probe may be an antibody, protein, bead, fluorophore, radioisotope, or a combination thereof. In one embodiment, the methylated mRNA consists essentially of a nucleic acid sequence having at least 95%, 96%, 97%, 98%, or 99% sequence identity to SEQ ID NO 8. In one embodiment, the methylated mRNA consists essentially of a probe coupled to a nucleic acid sequence having at least 95%, 96%, 97%, 98%, or 99% sequence identity to SEQ ID NO 8. In one embodiment, the methylated mRNA comprises a nucleic acid sequence of SEQ ID NO 8. In one embodiment, the methylated mRNA consists essentially of a nucleic acid sequence of SEQ ID NO 8.

In one embodiment, a methylated mRNA comprises a nucleic acid sequence having at least 95%, 96%, 97%, 98%, or 99% sequence identity to SEQ ID NO 9. In one embodiment, the methylated mRNA further comprises a probe, wherein the probe may be an antibody, protein, bead, fluorophore, radioisotope, or a combination thereof. In one embodiment, the methylated mRNA consists essentially of a nucleic acid sequence having at least 95%, 96%, 97%, 98%, or 99% sequence identity to SEQ ID NO 9. In one embodiment, the methylated mRNA consists essentially of a probe coupled to a nucleic acid sequence having at least 95%, 96%, 97%, 98%, or 99% sequence identity to SEQ ID NO 9. In one embodiment, the methylated mRNA comprises a nucleic acid sequence of SEQ ID NO 9. In one embodiment, the methylated mRNA consists essentially of a nucleic acid sequence of SEQ ID NO 9.

In one embodiment, a methylated mRNA comprises a nucleic acid sequence having at least 95%, 96%, 97%, 98%, or 99% sequence identity to SEQ ID NO 10. In one embodiment, the methylated mRNA further comprises a probe, wherein the probe may be an antibody, protein, bead, fluorophore, radioisotope, or a combination thereof. In one embodiment, the methylated mRNA consists essentially of a nucleic acid sequence having at least 95%, 96%, 97%, 98%, or 99% sequence identity to SEQ ID NO 10. In one embodiment, the methylated mRNA consists essentially of a probe coupled to a nucleic acid sequence having at least 95%, 96%, 97%, 98% or 99% sequence identity to SEQ ID NO 10. In one embodiment, the methylated mRNA comprises a nucleic acid sequence of SEQ ID NO 10. In one embodiment, the methylated mRNA consists essentially of a nucleic acid sequence of SEQ ID NO 10.

In one embodiment, a methylated mRNA comprises a nucleic acid sequence having at least 95%, 96%, 97%, 98%, or 99% sequence identity to SEQ ID NO 11. In one embodiment, the methylated mRNA further comprises a probe, wherein the probe may be an antibody, protein, bead, fluorophore, radioisotope, or a combination thereof. In one embodiment, the methylated mRNA consists essentially of a nucleic acid sequence having at least 95%, 96%, 97%, 98%, or 99% sequence identity to SEQ ID NO 11. In one embodiment, the methylated mRNA consists essentially of a probe coupled to a nucleic acid sequence having at least 95%, 96%, 97%, 98%, or 99% sequence identity to SEQ ID NO 11. In one embodiment, the methylated mRNA comprises a nucleic acid sequence of SEQ ID NO 11. In one embodiment, the methylated mRNA consists essentially of a nucleic acid sequence of SEQ ID NO 11.

In one embodiment, a methylated mRNA comprises a nucleic acid sequence having at least 95%, 96%, 97%, 98% or 99% sequence identity to SEQ ID NO 12. In one embodiment, the methylated mRNA further comprises a probe, wherein the probe may be an antibody, protein, bead, fluorophore, radioisotope, or a combination thereof. In one embodiment, the methylated mRNA consists essentially of a nucleic acid sequence having at least 95%, 96%, 97%, 98%, or 99% sequence identity to SEQ ID NO 12. In one embodiment, the methylated mRNA consists essentially of a probe coupled to a nucleic acid sequence having at least 95%, 96%, 97%, 98%, or 99% sequence identity to SEQ ID NO 12. In one embodiment, the methylated mRNA comprises a nucleic acid sequence of SEQ ID NO 12. In one embodiment, the methylated mRNA consists essentially of a nucleic acid sequence of SEQ ID NO 12. In one embodiment, a methylated mRNA comprises a biotin coupled to SEQ ID NO 12. In one embodiment, a methylated mRNA consists essentially of a biotin coupled to SEQ ID NO 12.

In one embodiment, a high-throughput method of testing lead compounds comprises providing an engineered protein, a methylated mRNA, and a lead compound. The method comprises providing a engineered protein comprising an amino acid sequence having at least 95% sequence identity to SEQ ID NO 2; providing a recombinant mRNA comprising a nucleic acid sequence having at least 95% sequence identity to SEQ ID NO 10, and a methylated adenosine; providing a lead compound; and analyzing the effect of the lead compound on the engineered protein's ability to demethylate the methylated adenosine of the recombinant mRNA.

In one embodiment, a high-throughput method of testing lead compounds comprises providing an engineered protein, a methylated mRNA, and a lead compound. The method comprises providing an engineered protein comprising an amino acid sequence having at least 95% sequence identity to SEQ ID NO 3; providing a recombinant mRNA comprising a nucleic acid sequence having at least 95% sequence identity to SEQ ID NO 10 and a methylated adenosine; providing a lead compound; and analyzing the effect of the lead compound on the engineered protein's ability to demethylate the methylated adenosine of the recombinant mRNA.

In one embodiment, a high-throughput method of testing lead compounds comprises providing an engineered protein, a methylated mRNA, and a lead compound. The method comprises providing an engineered protein comprising an amino acid sequence having at least 95% sequence identity to SEQ ID NO 2; providing a recombinant mRNA comprising a nucleic acid sequence having at least 95% sequence identity to SEQ ID NOs 12 which includes a methylated adenosine; providing a lead compound; and analyzing the effect of the lead compound on the engineered protein's ability to demethylate the methylated adenosine of the recombinant mRNA.

In one embodiment, a high-throughput method of testing lead compounds comprises providing an engineered protein, a methylated mRNA, and a lead compound. The method comprises providing an engineered protein comprising an amino acid sequence having at least 95% sequence identity to SEQ ID NO 3; providing a recombinant mRNA comprising a nucleic acid sequence having at least 95% sequence identity to SEQ ID NO 12 and a methylated adenosine; providing a lead compound; and analyzing the effect of the lead compound on the engineered protein's ability to demethylate the methylated adenosine of the recombinant mRNA.

In one embodiment, a high-throughput method of testing lead compounds comprises providing an engineered protein, a methylated mRNA, and a lead compound. The method comprises providing an engineered protein comprising an amino acid sequence having at least 95% sequence identity to SEQ ID NO 1; providing a recombinant mRNA comprising a nucleic acid sequence having at least 95% sequence identity to SEQ ID NO 12; providing a lead compound; and analyzing the effect of the lead compound on the engineered protein's ability to demethylate the methylated adenosine of the recombinant mRNA.

In one embodiment, a high-throughput method of testing lead compounds comprises providing an engineered protein having at least 95% sequence identity selected from the group consisting of SEQ ID NO 1, SEQ ID NO 2, SEQ ID NO 3, SEQ ID NO 4, SEQ ID NO 5, SEQ ID NO 6, and SEQ ID NO 7; providing a methylated mRNA comprising a nucleic acid sequence having at least 95% sequence identity selected from the group consisting of SEQ ID NO 8, SEQ ID NO 9, SEQ ID NO 10, SEQ ID NO 11, and SEQ ID NO 12; providing a lead compound, and analyzing the effect of the lead compound on the engineered protein's ability to remove the methyl group from the methylated mRNA. In some embodiments, the engineered protein, the methylated mRNA, the lead compound, or a combination thereof include a probe.

In one embodiment, the step of analyzing includes methods known to those of skill in the art. In one embodiment, the step of analyzing includes microscopy or electrophoretic mobility shift assay.

In one embodiment, the present disclosure is directed to a method of inducing proliferation in a cardiomyocyte cell. The method comprises providing a demethylase to the cardiomyocyte cell, wherein the demethylase is designed to remove a methyl group from Region 2 of a Bmp10 mRNA. In one embodiment, the demethylase comprises an amino acid sequence having at least 95% sequence identity to SEQ ID NO 2. In one embodiment, the demethylase comprises an amino acid sequence having at least 95% sequence identity to SEQ ID NO 3. In one embodiment, the demethylase comprises an amino acid sequence having at least 95% identity to SEQ ID NO 4. In one embodiment, the demethylase comprises an amino acid sequence having at least 95% identity to SEQ ID NO 5.

In one embodiment, the demethylase is designed to remove a methyl group from site M2+M3 of Region 2. In one embodiment, the site M2+M3 comprises the nucleic acid sequence having at least 95% sequence identity to SEQ ID NO 10. In one embodiment, the demethylase is designed to remove a methyl group from site M2, wherein site M2 comprises SEQ ID NO: 11. In some embodiments, the demethylase is an engineered protein comprising a probe and having at least 95% sequence identity to SEQ ID NO 2, or SEQ ID NO 3.

In one embodiment, the demethylase comprises an amino acid sequence having at least 95% sequence identity to SEQ ID NO 3 and is designed to remove a methyl group from Region 2 of Bmp10 mRNA comprising a nucleic acid sequence having at least 95% sequence identity to SEQ ID NO 10. In some embodiments, the demethylase comprises an amino acid sequence having at least 95% sequence identity to SEQ ID NO 2 or SEQ ID NO 3 and is designed to remove a methyl group from adenosine of Bmp10 mRNA.

In one embodiment, the demethylase comprises an amino acid sequence having at least 95% sequence identity to SEQ ID NO 4 and is designed to remove an m⁶A from Region 2, wherein Region 2 comprises a nucleic acid sequence having at least 95% sequence identity to SEQ ID NO 10.

In one embodiment, the demethylase comprises an amino acid sequence having at least 95% sequence identity to SEQ ID NO 5 and is designed to remove an m⁶A from Region 2, wherein Region 2 comprises a nucleic acid sequence having at least 95% sequence identity to SEQ ID NO 10.

In one embodiment, a method for inducing proliferation in a cardiomyocyte cell is provided. The method comprises providing an m⁶A methylation inhibitor to the cardiomyocyte cell in an amount effective to remove a methyl group from Region 2 of Bmp10 mRNA. In one embodiment, the m⁶A methylation inhibitor comprising 3-DZA. In one embodiment, the m⁶A methylation inhibitor consists essentially of 3-DZA.

In one embodiment a method of enhancing proliferation of cardiomyocytes is provided. The method comprises contacting the cardiomyocytes with an effective amount of a pharmaceutical composition to remove a methyl group from Region 2 of Bmp10 mRNA, wherein the pharmaceutical composition includes a demethylase and a pharmaceutically acceptable carrier. In some embodiments, the demethylase is an engineered protein comprising a sequence having at least 95% sequence identity selected from a group consisting of SEQ ID NO 1, SEQ ID NO 2, SEQ ID NO 3, SEQ ID NO 4, and SEQ ID NO 5. In some embodiments, the methyl group is bound to an adenosine of Region 2.

In one embodiment, a method of culturing cardiomyocyte cells is provided. The method comprises introducing a pharmaceutical composition into a cardiomyocyte culture medium, wherein the pharmaceutical composition includes a demethylase and pharmaceutically acceptable carrier. In some embodiments, the demethylase is an engineered protein comprising a sequence having at least 95% sequence identity selected from a group consisting of SEQ ID NO 1, SEQ ID NO 2, SEQ ID NO 3, SEQ ID NO 4, and SEQ ID NO 5.

In one embodiment a culture medium is provided the culture medium comprises a buffer, and a pharmaceutical composition that includes a demethylase and a pharmaceutically acceptable carrier, wherein the amount of demethylase present in the culture medium is effective to remove a methyl group from Region 2 of Bmp10 mRNA when in contact with a cardiomyocyte. In some embodiment, the demethylase is an engineered protein comprising a sequence having at least 95% sequence identity selected from a group consisting of SEQ ID SEQ ID NO 1, SEQ ID NO 2, SEQ ID NO 3, SEQ ID NO 4, and SEQ ID NO 5. In some embodiments, the demethylase further comprises a means of transporting across the memebrane of a cardiomyocyte. Means of transport are known to those skilled in the art and include but are not limited to, peptides, liposome, nanoparticles, and viruses. In some embodiments, the peptide is able to transport the demethylase from the culture medium into the cytosol and nucleus of the cardiomyocyte.

In one embodiment, a method of producing a culture medium is provided. The method comprises: mixing a buffer with a demethylase, wherein the amount of demethylase present in the buffer is effective to remove a methyl group from Region 2 of Bmp10 mRNA when the demethylase is in contact with a cardiomyocyte. In some embodiment, the demethylase is an engineered protein comprising a sequence having at least 95% sequence identity selected from a group consisting of SEQ ID NO 1, SEQ ID NO 2, SEQ ID NO 3, SEQ ID NO 4, and SEQ ID NO 5.

In accordance with embodiment 1, a method of promoting cardiomyocyte cell proliferation is provided. The method comprises providing a demethylase to the cardiomyocyte, wherein the demethylase is designed to remove a methyl group from Region 2 of the Bmp10 mRNA.

In accordance with embodiment 2, the method of embodiment 1 is provided wherein the demethylase comprises an amino acid sequence having at least 95% sequence identity to Brg1 helicase domain SEQ ID NO 3.

In accordance with embodiment 3, the method of embodiment 1 is provided wherein the demethylase comprises an amino acid sequence having 95% identity to SEQ ID NO 4.

In accordance with embodiment 4, the method of embodiment 1 is provided wherein the demethylase comprises an amino acid sequence having at least 95% identity to SEQ ID NO 5.

In accordance with embodiment 5, the method of embodiment 1 is provided wherein the demethylase is designed to remove a methyl group from site M2+M3 of Region 2.

In accordance with embodiment 6, the method of embodiment 5 is provided wherein site M2+M3 comprises the nucleic acid sequence having at least 95% sequence identity to SEQ ID NO 10.

In accordance with embodiment 7 the method of embodiment 6 is provided wherein site M2 comprises of SEQ ID NO 11.

In accordance with embodiment 8, the method of embodiment 1 is provided wherein the demethylase comprises an amino acid sequence having at least 95% sequence identity to SEQ ID NO 3 and Region 2 comprises a nucleic acid sequence having at least 95% sequence identity to SEQ ID NO 10.

In accordance with embodiment 9, the method of embodiment 1 is provided wherein the demethylase comprises an amino acid sequence having at least 95% sequence identity to SEQ ID NO 4 and Region 2 comprises a nucleic acid sequence having at least 95% sequence identity to SEQ ID NO 10.

In accordance with embodiment 10, the method of embodiment 1 is provided wherein the demethylase comprises an amino acid sequence having at least 95% sequence identity to SEQ ID NO 5 and Region 2 comprises a nucleic acid sequence having at least 95% sequence identity to SEQ ID NO 10.

In accordance with embodiment 11, a method of inducing proliferation in a cardiomyocyte cell is provided. The method comprises providing an m⁶A methylation inhibitor to the cardiomyocyte cell.

In accordance with embodiment 12, the method of embodiment 11 is provided wherein the m⁶A methylation inhibitor comprising 3-DZA.

In accordance with embodiment 13, a method of inducing proliferation in a cardiomyocyte cell is provided. The method comprises providing a recombinant protein comprising a demethylase to the cell, wherein the demethylase enhances the Brg1 demethylation activity, wherein the recombinant protein comprises an amino acid sequence having at least 95% sequence identity to SEQ ID NO 2,wherein the Brg1 demethylation activity is enhanced relative to the base line level of Brg1.

In accordance with embodiment 14, the method of embodiment 13 is provided wherein the recombinant protein comprises an amino acid sequence having at least 95% sequence identity to SEQ ID NO 3.

In accordance with embodiment 15, the method of embodiment 13 is provided wherein the Brg1 helicase domain comprises an amino acid sequence having at least 95% sequence identity to SEQ ID NO 4.

In accordance with embodiment 16, the method of embodiment 13 is provided wherein the Brg1 helicase domain comprises an amino acid sequence having at least 95% sequence identity to SEQ ID NO 5.

In accordance with embodiment 17, a method of testing lead compounds in a high-throughput assay is provided. The method comprises providing a recombinant protein comprising an amino acid sequence having at least 95% sequence identity to SEQ ID NO 2;
providing a recombinant mRNA comprising a nucleic acid sequence having at least 95% sequence identity to SEQ ID NO 10 and a methylated adenosine;
providing a lead compound; and
analyzing the effect of the lead compound on the recombinant protein's ability to demethylate the methylated adenosine of the recombinant mRNA.

In accordance with embodiment 18, a method of enhancing proliferation of cardiomyocytes is provided. The method comprises contacting the cardiomyocytes with an effective amount of a pharmaceutical composition to remove a methyl group from Region 2 of Bmp10 mRNA, wherein the pharmaceutical composition includes a demethylase and a pharmaceutically acceptable carrier.

In accordance with embodiment 19, the method of embodiment 18 is provided wherein the demethylase comprises an amino acid sequence having at least 95% sequence identity to SEQ ID NO 4.

In accordance with embodiment 20, the method of embodiment 18 is provided wherein the demethylase comprises an amino acid sequence having at least 95% sequence identity to SEQ ID NO 5.

In accordance with embodiment 21, the method of embodiment 18 is provided wherein the demethylase is designed to remove a methyl group from site M2+M3 of Region 2.

In accordance with embodiment 22, the method of claim 18 is provided wherein site M2+M3 comprises the nucleic acid sequence having at least 95% sequence identity to SEQ ID NO 10.

In accordance with embodiment 23, the method of embodiment 22 is provided wherein site M2+M3 consists essentially of SEQ ID NO 11.

In accordance with embodiment 24, the method of embodiment 18 is provided wherein the demethylase comprises an amino acid sequence having at least 95% sequence identity to SEQ ID NO 3 and Region 2 comprises a nucleic acid sequence having at least 95% sequence identity to SEQ ID NO 10.

In accordance with embodiment 25, the method of embodiment 18 is provided wherein the demethylase comprises an amino acid sequence having 95% sequence identity to SEQ ID NO 4 and Region 2 comprises a nucleic acid sequence having at least 95% sequence identity to SEQ ID NO 10.

In accordance with embodiment 26, the method of embodiment 18 is provided wherein the demethylase comprises an amino acid sequence having at least 95% sequence identity to SEQ ID NO 5 and Region 2 comprises a nucleic acid sequence having at least 95% sequence identity to SEQ ID NO 10.

In accordance with embodiment 27, a method of culturing cardiomyocyte cells is provided. The method comprises introducing a pharmaceutical composition into a cardiomyocyte culture medium, wherein the pharmaceutical composition includes a demethylase and pharmaceutically acceptable carrier.

In accordance with embodiment 28, the method of embodiment 27 is provided wherein, the pharmaceutical composition is introduced in an amount to effectively remove a methyl group from Region 2 of Bmp10 mRNA.

In accordance with embodiment 29, a culture medium is provided. The culture medium comprises:
a buffer, and
a pharmaceutical composition that includes a demethylase and a pharmaceutically acceptable carrier, wherein the amount of demethylase present in the culture medium is effective to remove a methyl group from Region 2 of Bmp10 mRNA when in contact with a cardiomyocyte.

In accordance with embodiment 30, a method of producing a culture medium is provided. The method comprises: mixing a buffer with a demethylase, wherein the amount of demethylase present in the buffer is effective to remove a methyl group from Region 2 of Bmp10 mRNA when the demethylase is in contact with a cardiomyocyte.

In accordance with embodiment 31, an engineered protein is provided. The embodiment comprises an amino acid sequence having an amino acid sequence having at least 95% sequence identity to SEQ ID NO 2.

All amino acid sequences are intended to include their respective nucleic acid sequences, and all nucleic acid sequences, when appropriate, are intended to include their respective amino acid sequences. All nucleic acid sequences are intended to include their complementary nucleic acid sequences.

The following Examples are provided to expand and further support the above disclosed embodiments. The Examples are not meant to be limiting. Further, any reference or patent application mentioned in this specification are each hereby incorporated in their entirety.

### EXAMPLE 1: MICE

CD1 male and female mice were purchased from Charles River (Strain Code: 022). Sm22αCre¹⁻³ and Brg1^{fl/fl} mice were generated similar to the protocol described in Sumi-Ichinose, C., Ichinose, H., Metzger, D. & Chambon, P. SNF2beta-BRG1 is essential for the viability of F9 murine embryonal carcinoma cells. Mol Cell Biol 17, 5976-5986 (1997). The date of observing of a vaginal plug was set as embryonic day E0.5 by convention.

### EXAMPLE 2: CELL CULTURE, SIRNA KNOCKDOWN, AND PLASMID TRANSFECTION

293T, H1299, and SW13 cell lines were purchased from ATCC. Cells were maintained in Dulbecco's Modified Eagle's Medium (DMEM) with 10% fetal bovine serum (PBS) and 1% 100× Pen/Strep (Gibco). Mouse embryonic ventricular cardiomyocytes were cultured using acceptable protocols. Briefly, ventricles of E18.5 CD1 mouse hearts were excised and digested with type II collagenase 4 or 5 times, for about 15 minutes each. Cells were then collected and resuspended in DMEM with 10% FBS. Cells were plated for about 1 hour to allow the attachment and the removal of non-myocyte cells. Cardiomyocytes were then plated at a density of about 2×10⁵/ml and cultured for further experiments. Gene knockdown experiments were conducted using Brg1 siRNA (Santa Cruz, sc-29827), YTHDF2 siRNA (ThermoFisher Scientific, s102905), or negative control siRNA (QIAGEN, 1027281). Transfections were achieved using Lipofectamine RNAiMAX (Invitrogen) for siRNA, Lipofectamine 2000 for single plasmid transfection, or Lipofectamine LTX Plus (Invitrogen) for co-transfection of two or more types of plasmids assay, according to manufacturer's protocols.

### EXAMPLE 3: M⁶A DOT BLOT ASSAY

RNA samples were quantified using NanoDrop™ 2000c Spectrophotometers (Thermo Fisher Scientific), and equal amounts were spotted onto positively charged nylon membranes (Sigma-Aldrich, 11209299001). The membranes were then UV crosslinked, blocked, and exposed to rabbit anti-m⁶A antibody (Synaptic System Cat. No. 202003) at 4° C. overnight. Membranes were then washed in 0.1% PBST, followed by incubation in secondary antibody (HRP-conjugated mouse anti-rabbit IgG, diluted 1:20000 in block) for about 1 hour (hr) at about Room Temperature (RT). Membranes were again washed in 0.1% PBST and developed with SuperSignal ECL Western blot detection kits (Pierce, 34095) using LI-COR odyssey image system.

### EXAMPLE 4: TWO-DIMENSION THIN LAYER CHROMATOGRAPHY (2D-TLC) ASSAY

2D-TLC was performed. 100 to 200 ng of polyA+ RNAs purified with PolyATtract™ mRNA Isolation Systems IV (Promega, Z5310) were digested using 2000 units of RNAse T1 (ThermoFisher Scientific, FEREN0541) in a final volume of 25 μl in 1× PNK (70 mM Tris-HCl, 10 mM MgCl₂, 5 mM DTT, pH 7.6) (NEB, B0201S) buffer incubated at about 37° C. for about 1 hour. The RNAs were labeled with 10 units of PNK and 1 μl [ [symbol] -³²P] ATP (6000 Ci/mmol, Perkin-Elmer). The reactions were cleaned with a G25 column, and RNAs precipitated with ethanol, re-suspended in 5 μl of 50 mM sodium acetate (pH 5.5), and digested with 1 unit of nuclease P1 (Sigma-Aldrich, N8630-1VL). 1 μl was loaded on a cellulose TLC glass plate (EMD chemicals, 5716-7). The first dimension was resolved in isobutyric acid: 0.5 M NH₄OH (5:3, v/v), and the second dimension resolved in isopropanol:HCl:water. The plates were exposed on a phosphor screen and scanned on a Bio-Rad imaging system.

### EXAMPLE 5: M⁶A-RNA-IP

m⁶A RNA immunoprecipitation was conducted as described with some modifications. Total RNA was isolated using TRIzol reagent. Polyadenylated RNAs (polyA+ RNAs) were enriched from total RNAs using one round of PolyATtract® mRNA Isolation Systems (Promega) followed by further removal of contaminated rRNA using RiboMinus Eukaryote Kit v2 (Ambion). mRNA samples were chemically fragmented into ˜100-nucleotide-long fragments by 5 minutes of incubation at 94° C. in NEBNext® Magnesium RNA Fragmentation buffer (Cat. E6150S). Fragmentation reactions were terminated with RNA Fragmentation Stop Solution, followed by RNA purification using Clean & Concentrator kit (Zymo Research Cat. No. R1017). 20 ng of fragmented mRNAs were saved as input, whereas 200 ng of fragmented mRNAs were incubated with 2 μg of anti-m⁶A antibody (Synaptic System Cat. No. 202003) in 1× IP buffer (10 mM Tris-HCl, pH 7.4, 150 mM NaCl, and 0.1% NP-40) on a rotating wheel overnight at 4° C. The m⁶A-RNA mixture was then incubated with 20 μl Protein G (Bio-Rad Cat. No.161-4023) for 2 hours at 4° C. on a rotating wheel. After washing three times (15 minutes each) with 1× IP buffer, bound mRNAs were eluted using 100 μl elution buffer containing 6.7 mM N6-Methyladenosine 5′-monophosphate sodium salt (Sigma-Aldrich, M2780) in 1× IP buffer, followed by mRNA recovery using RNA Clean & Concentrator kit (Zymo Research Cat. No. R1017). Recovered mRNAs were used for reverse transcription reaction, real-time PCR, and cDNA library construction for high-throughput sequencing.

### EXAMPLE 6: REAL-TIME QUANTITATIVE PCR (RT-QPCR)

RT-qPCR was performed to assess the relative abundance of mRNAs. The RT-PCR primers were designed to span exon-exon junctions to eliminate amplifications of genomic DNA and unspliced mRNAs. Reverse transcription was performed by PrimeScript™ RT reagent Kit (Clontech, RR037A). Real-time PCR reactions were performed using Power SYBR Green PCR Master Mix (Thermo Fisher Scientific, 4367659) with an Applied Biosystems realplex, and the primer sets were tested to be quantitative. Transcription factor IIb (TFIIb) was used as normalizer. Threshold cycles and melting curve measurements were performed with ABI software. P-values were calculated by the Student-t test. Error bars indicate standard error of mean.

List of primers used in the Examples.

Bmp10 R1-F:
(SEQ ID NO 13)
CTTCACGGAGCAAGATGGTA;
Bmp10 R1-R:
(SEQ ID NO 14)
TGTACTCTGGTGGATCCACT;
Bmp10 R2-F:
(SEQ ID NO 15)
CCATGAAGAGGTCGTCAT;
Bmp10 R2-R:
(SEQ ID NO 16)
TGCTCCTCTCCTCCTCGCT;
Bmp10 R3-F:
(SEQ ID NO 17)
CTGATGACCAAAGCAATGAC;
Bmp10 R3-R:
(SEQ ID NO 18)
AGAGCCTCTTCATCGGGCCCA;
Bmp10 R4-F:
(SEQ ID NO 19)
ATGATTCGTCCGCTCGGATCA;
Bmp10 R4-R:
(SEQ ID NO 20)
CGGCACTCATAGGCTTCATA;
Bmp10 q-F:
(SEQ ID NO 21)
CTCAAGACGCTGAACTTGTCG;
Bmp10 q-R:
(SEQ ID NO 22)
GAGAGGATATTTCCGGAGCCC;

### m⁶A-IP-Seq

Sequencing was carried out on Ion Proton System (ThermoFisher Scientific) according to manufacturer's instructions, using Ion Total RNA-Seq Kit v2 (ThermoFisher Scientific) for library construction and deep sequencing. Sequencing depth is about 10 million reads per sample.

### EXAMPLE 7: DETECTION OF M⁶A SITES AND M⁶A MOTIF ANALYSIS

UCSC mm10 canonical Known Genes are used to map reads. WinScore was computed using acceptable methods, and windows with WinScore >=2 (four-fold change in IP vs Control) were merged as putative peaks. Two replicates were combined to get Ctrl m⁶A peaks and Brg1 knockout (KO) m⁶A Peaks (IP vs Input). In addition, Brg1 KO specific peaks were identified using WinScore with Brg1 KO vs Ctrl, and Ctrl specific peaks were identified using WinSore with Ctrl vs. Brg1 KO. To identify motif search method, we took the top 2000 WT peaks, used negative peaks (WinScore <1) as background, and used homer program to search for motifs with 5, 7, or 9 bases.

### EXAMPLE 8: RNA IMMUNOPRECIPITATION

RNA immunoprecipitation (RNA-IP, RIP) was conducted. Briefly, E10.5 hearts or SW13 cells were crosslinked and lysed with lysis buffer (10 mM HEPES pH 7.5, 85 mM KCl, 0.5% NP-40, 1 mM dithiothreitol (DTT), 1× protease inhibitor) for tissues or lysis buffer (10 mM Tris-HCl pH 8.1, 10 mM NaCl, 1.5 mM MgCl2, 0.5% NP-40, 1 mM DTT, 1× protease inhibitor) for cells. Nuclei were isolated and sonicated using Bioruptor (Diagenode) (30 s on, 30 s off, power setting H, 5 min, performed twice) in nuclear lysis buffer (50 mM Tris-HCl pH 8.1, 150 mM NaCl, 0.1% NP-40, 1 mM DTT, protease inhibitor, ribonuclease inhibitor). The nuclear extract was collected then incubated with anti-Ythdf2 antibody (EMD Millipore ABE542, 1:100) and normal IgG control at 4° C. overnight together with Manga ChIP Protein G Magnetic Beads (Millipore). The beads were washed by wash buffer I (20 mM Tris-Hcl pH 8.1, 150 mM NaCl, 1% Triton X-100 and 0.1% SDS) three times, and wash buffer II (20 mM Tris-Hcl pH8.1, 500 mM NaCl, 1% Triton X-100 and 0.1% SDS) three times. Beads were then resuspended in 150 ml 150 mM RIPA (50 mM Tris pH 7.5, 150 mM NaCl, 1 mM EDTA, 0.1% SDS, 1% NP-40, 0.5% sodium deoxycholate) with 5 ml Proteinase K and incubated for 1 h at 65° C. We added 1 ml of TRIzol to the sample, and RNA was extracted using the Quick-RNA Mini Kit with the on-column DNase I digest (Zymo Research). RT and qPCR were then conducted with the purified RNA.

### EXAMPLE 9: WESTERN BLOT ANALYSIS

Cells were collected and washed once with ice-cold phosphate-buffered saline (PBS) and pellets were resuspended in RIPA lysis buffer (0.5 M Tris-HCl, pH 7.4, 1.5 M NaCl, 2.5% deoxycholic acid, 10% NP-40, 10 mM EDTA). After sonication and centrifugation, the supernatants were collected and boiled. The blots were reacted with antibodies for YTHDF2 (EMD Millipore ABE542, 1:1,000 WB); Brg1 (Santa Cruz sc-17796, 1:1,000 WB); FTO (Abcam ab92821, 1:1,000 WB); METTL3 (Abnova H00056339-B01P, 1:1,000 WB); METTL14 (Sigma-Aldrich HPA038002, 1:1,000 WB), ALKBH5 (Sigma-Aldrich HPA007196, 1:1,000 WB), and GAPDH (Santa Cruz sc-25778, 1: 2000) followed by horseradish peroxidase (HRP)-conjugated anti-mouse IgG or HRP-conjugated anti-rabbit IgG (Jackson). Chemiluminescence was detected with with SuperSignal ECL Western blot detection kits (Pierce, 34095) using LI-COR odyssey image system.

### EXAMPLE 10: M⁶A INDIVIDUAL-NUCLEOTIDE-RESOLUTION CROSS-LINKING AND IMMUNOPRECIPITATION (MICLIP)

miCLIP was performed. Briefly, total RNAs extracted from embryonic hearts were fragmented using fragmentation reagent (Life Technologies) to a size between 90 and 200 nt. After stopping the reaction, the fragmented RNA pool was transferred to IP buffer (50 mM Tris pH 7.4, 100 mM NaCl, 0.05% NP40) and incubated with 5-10 ug anti-m⁶A antibody from Abcam (ab151230) for 2 h at 4° C. rotating head over tail. After 2 h, the mixture of RNA and antibody was transferred into a 3-cm cell culture dish and crosslinked twice with 0.15 J cm⁻² UV light (254 nm) in a Stratalinker (Agilent). RNAs immunoprecipitated by m⁶A antibody were isolated and reverse-transcribed, and a 79bp fragment from Bmp10 R2 region and covering M2 sites was PCR-amplified and cloned into TOPO vector for sequencing. RNAs were pooled from 10 to 12 E10.5 hearts or from 6 to 8 E12.5 hearts for one single miCLIP experiment. Crosslink-induced mutations at position 0, +1 and +2 relative to the m⁶A introduced during reverse transcription were analyzed to determine positions of m⁶A sites. Bmp10 Region2 79nt primer F: ATGAAGAGGTCGTCATGGCT (SEQ ID NO 23); R: CGGTCCACGCCATCATACAT (SEQ ID NO 24). 79nt is part of Bmp10 Region 2(140nt) and covers M2 sites.

### EXAMPLE 11: MAZF DIGESTION AND QPCR ASSAY

MazF-qPCR was performed. Briefly, mRNAs isolated from embryonic hearts were incubated with or without MazF (Clontech, 2415A) enzyme digestion in the reaction buffer at 37° C. for about 30 min. After digestion, mRNAs were purified by RNA Clean & Concentrator-5 kit (ZYMO Research) and reverse-transcribed into cDNAs for qPCR to quantitate methylation of the first adenosine of the ACA motif.

### EXAMPLE 12: ATPASE ASSAY

ATPase activity was determined using an ATPase Colorimetric Assay Kit (Catalogue no. 601-0120, Novus Biologicals). Briefly, 100 μl of 40 nM (4 pmol) purified protein was mixed with 100 μl of the reaction buffer (the final concentration is 20 nm purified protein, 50 mM Tris, 2.5 mM MgCl2, and 0.5 mM ATP), incubating for about 30 minutes at Room Temperature (RT). The reaction is then stopped by adding 50 ul of Gold mix. After 2 minutes, the stabilizer is added and mixed thoroughly. The ATPase activity was calculated by measurement of liberated inorganic phosphate (Pi) at a wavelength in the range 590-660 nm and the standard curve.

### EXAMPLE 13: MRNA STABILITY ASSAY

Mouse E18.5 cardiomyocytes with Control, Brg1, or YTHDF2 siRNA knockdown were treated with actinomycin D (1 μg/ml) (Sigma, A9415) from 0-4 hr. RNA extraction and RT-qPCR were performed as described above.

### EXAMPLE 14: 4-THIOURACIL (4tU) PULSE AND URACIL CHASE ASSAY

The 4tU was performed. Briefly, E17-E18 embryonic cardiomyocytes were isolated and cultured overnight. RNAi transfection was then performed, and cells were cultured for 48 hrs. 4-Thiouracil (4tU) (100 μM, Sigma, 440736) was added to the fresh media and cultured for 12 hrs. The culture media was then replaced by new media with uracil (12 mM, Sigma, U1128) and cultured until cell collection at times indicated. Total RNAs were then isolated with TRIzol (ThermoFisher Scientific) and biotinylation of 4tU-labeled RNA was performed using (N-(6-(Biotinamido)hexyl)-3′-(2′-pyridyldithio)-propionamide (EZ-Link Biotin-HPDP, ThermoFisher Scientific). Biotin-labeled 4tU incorporated RNA was then pulled down by Dynabeads® MyOne™ Streptavidin C1 (ThermoFisher Scientific) and eluted for reverse transcription (SuperScript™ IV VILO™, ThermoFisher Scientific). Bmp10 was quantitated by qPCR (StepOnePlus Real-Time PCR System, ThermoFisher Scientific).

### EXAMPLE 15: 3-DEAZAADENOSINE TREATMENT

3-Deazaadenosine (3-DZA, D8296, Sigma-Aldrich) was injected intraperitoneally of about 2 mg/kg/day from embryonic day 7.5 to 10.5.

### EXAMPLE 16: HISTOLOGY AND BRDU STAINING

Histology of embryonic hearts was performed as described. Embryos were fixed overnight in 4% PFA in PBS. They were then dehydrated through an ethanol series, treated with xylenes, and embedded in paraffin wax overnight with several changes. Embryos were oriented for transverse sections and cut in 7 μm sections using a Leica microtome. Following re-hydration, the sections were consecutively stained with Hematoxylin & Eosin (Sigma-Aldrich, St. Louis, Mo.), dehydrated, and mounted in Permount (Sigma) prior to imaging. The thickness of compact myocardium was measured at 20 points along the circumference of the left ventricle, and the results were presented by normalizing with the compact myocardium thickness in the Ctrl and Vehicle group. Pregnant mice were injected with BrdU (Sigma, 100 μg/g, intraperitoneal injection) for 6 hours prior to embryo isolation at E10.5. Incorporated BrdU was stained in the tissue-section of the heart according to the manufacturer's protocol (Invitrogen, BrdU Staining Kit 93-3943). BrdU incorporation was quantitated by the percentage of BrdU-positive cells in the myocardium. p-values were calculated by the Student-t test. Error bars indicate one standard deviation.

### EXAMPLE 17: CARDIAC TROPONIN T AND PHOSPHO-HISTONE H3 CO-STAINING

E10.5 hearts was collected and embedded in paraffin. After hydration and antigen retrieval, E10.5 heart was blocked with PBS containing 5% NGS, and stained with Cardiac Troponin T (Abcam, Cat. No. ab8295, 1: 200) and phospho-Histone H3 (Cell Signaling Technology, Cat. No. 9701, 1:100) antibodies in blocking solution, followed by secondary antibodies conjugated to Alexa488 and Alexa594 (Invitrogen). E10.5 hearts were photographed by confocal microscope (Leica TCS SP8). Phospho-Histone H3 positive Cardiomyocytes were quantitated by normalizing to cardiomyocytes in the myocardium. p-values were calculated by the Student-t test.

### EXAMPLE 18: M⁶A IMMUNOSTAINING

Cells transfected with different Brg1 constructs were fixed with 2.5% paraformaldehyde (PFA) in phosphate-buffered saline (PBS) for 30 minutes, permeabilized with 0.5% Triton X-100 for 1 hour and treated with 2N HCl for 1 hour. Cells were then blocked with PBS containing 5% NGS, and stained with Brg1 (Santa Cruz sc-374197X, 1:500) and m⁶A (Synaptic System Cat. No. 202003, 1:50) antibodies in blocking solution, followed by secondary antibodies conjugated to Alexa488 and Alexa594 (Invitrogen). Cells were photographed by confocal microscope (Leica TCS SP8). The fluorescence intensity of Brg1-transfected cells was analyzed by Image J software.

### EXAMPLE 19: PROTEIN EXPRESSION AND PURIFICATION

Expression and purification of MBP fusion proteins were performed. Mouse Brg1 DExx-box domain (D1) (Amino acids 774-913 of Brg1), Helicase-C domain (D2) (Amino acids 1086-1204 of Brg1), the entire helicase region (D1D2) (774-1204) as well as the D1D2 domains of snf2 (790-1212) were amplified by PCR and cloned into pMAL vector. Construct mutations were achieved by GeneArt® Site-Directed Mutagenesis kit (ThermoFisher Scientific, A13282). MBP fusion proteins were induced by IPTG and purified by Amylose resin (E8021S, NEB).

### EXAMPLE 20: BIOCHEMICAL ASSAY OF M⁶A DEMETHYLATION ACTIVITY IN VITRO

The demethylation activity assay was performed. In brief, the assay was performed in standard 100 μl of reaction mixture containing RNA with m⁶A, purified protein, 2.83 mM of (NH₄)₂Fe(SO₄)₂·6H₂O, 300 μM of α-KG, 20 mM of L-ascorbic acid, 50 μg ml⁻¹ of BSA, and 50 mM of HEPES buffer (pH 7.0). The reaction was incubated at room temperature, quenched by addition of 5 mM EDTA and heating for 5 min at 95° C., followed by m⁶A dot blot analysis.

### EXAMPLE 21: NUCLEOSOME ASSEMBLY ASSAY

Nucleosomes were assembled using EpiMark Nucleosome Assembly Kit (E5350S, NEB) following the manufacturer's instruction with minor modifications. In brief, recombinant human core histone octamers (2:1 mix of histone H2A/H2B dimer and histone H3.1/H4 tetramer) were mixed with purified double-stranded DNA of Myh6 core promoter (202 bp, −428 to −227) amplified by primers of CAGATAGCCAGGGTTGAAAG and TGGGCAGATAGAGGAGAGAC at the molecular ratio of 1:1. The salt concentration was gradually lowered by dilution to allow the formation of nucleosomes. After nucleosome assembly, recombinant proteins (1.25 pmol of MBP, Brg1-D1D2 and D1D2 mutants) were mixed with 1.25 pmol nucleosomes in the presence of ATP (2 mM). The mixtures were placed on ice for 15 min and then analyzed with 1% agarose gel.

### EXAMPLE 22: LIQUID CHROMATOGRAPHY-TANDEM MASS SPECTROMETRY (LC-MS/MS)

Cell mRNA for LC-MS/MS: 200 ng of mRNA was digested by nuclease P1 (2 U) in 25 μl of buffer containing 10 mM of NH₄OAc (pH=5.3) at 42° C. for 2 h, followed by the addition of NH₄HCO₃ (1 M, 3 μl, freshly made) and alkaline phosphatase (0.5 U). After an additional incubation at 37° C. for 2 h, sample was diluted to 50 μl and filtered (0.22 μm pore size, 4 mm diameter, Millipore), and 5 μl of the solution was injected into LC-MS/MS.

RNA probe for LC-MS/MS: After in vitro biochemistry reaction, all samples were inactivated by adding 1 μl EDTA (0.5 M) and heated at 95° C. for 5 min, following by adding 0.5 μl proteinase K (20 mg/ml) into each reaction at 56° C. for at least 15 min to completely digest the precipitated protein. The mixture was treated with 72° C. for 15 min to inactivate proteinase K. The reacted nucleic acid substrates (1 μg) were enzymatically digested into single nucleoside by mixture with 1 U nuclease P1 (Sigma) and 1 U alkaline phosphatase (NEB). Nucleosides were separated by reverse phase ultra-performance liquid chromatography (LC) on a C18 column with on-line mass spectrometry detection using Finnigan LTQ-Orbitrap hybrid tandem mass spectrometer (Thermo Fisher Scientific) in positive electrospray ionization mode. The nucleosides were quantified by using the nucleoside to base ion mass transitions of 282 to 150 (m⁶A), and 268 to 136 (A). Quantification was performed in comparison with the standard curve obtained from pure nucleoside standards running on the same batch of samples. The ratio of m⁶A to A was calculated based on the calibrated concentrations.

### EXAMPLE 23: RNA ELECTROPHORETIC MOBILITY SHIFT ASSAY (EMSA) AND KD CALCULATION

Biotin-labelled RNA control probe:
Biotin—5′UCGUCAUGGCUGAACUGCGGUUGUACACGCUGGUGCAGAGAGAUCGCAU-3′ (SEQ ID NO 25); and Biotin-labelled RNA probe with m⁶A:
Biotin—5′UCGUCAUGGCUGAACUGCGGUUGU(m⁶A)CACGCUGGUGCAGAGAGAUCGCAU-3′ (SEQ ID NO 26); were synthesized by Gene Link (Orlando, Fla.). EMSA was performed by using the LightShift™ Chemiluminescent RNA EMSA Kit (Cat. 20158, Thermo Scientific). The labelled probe was incubated with appropriate amounts of recombinant proteins in 10 μl in the 1× binding buffer (10 mM HEPES-KOH, pH 7.3, 10 mM NaCl, 1 mM MgCl2, 1 mM DTT) with 5 μg tRNA carrier at room temperature for 30 min. The reactions were then loaded onto 1% 0.5× TBE agarose gel and transferred to Bright Star Plus positive charged membrane. The biotin-labelled probes were detected and quantified subsequently using Odyssey Infrared Imaging System. The shifted signals were quantified and plotted against amount of the MBP-D1D2 protein ((SEQ ID NO 27) coupled to (SEQ ID NO 2)) with GraphPad Prism (GraphPad). The software facilitates the fitting of nonlinear regression model and calculation of Kd values based on the fitting curve. The errors and r² values were also generated from the fitting curve.

### EXAMPLE 24: BRG1 R973Q INDUCIBLE POINT MUTATION MOUSE LINE

The Brg1 R973Q inducible point mutation knock-in mouse line was generated by ES cell gene targeting (Data not shown). The targeting vector was constructed by recombination. The 5′ distal LoxP site was inserted 192 bp upstream of Brg1 exon20 in forward direction. The FRT-flanked Neo cassette was placed 214 bp downstream of exon20, which was followed by the engineered inversion cassette containing duplicated exon20 with the specific point mutation (CGG>CAG, R973Q) and their flanking genomic sequences for correct splicing. The inversion cassette was flanked by mutant Lox71/Lox66 sites. The long homology arm was extending about 6.2 kb to the 5′ distal LoxP site, and the short homology arm was about 2 kb 3′ to the inversion cassette. The targeting vector was then linearized and electroporated into mouse embryonic stem (ES) cells. Neomycin-resistant clones were tested for correct gene targeting by Southern Blot. Following verification of correct targeting and karyotypes, two positive ES clones were expanded and injected into blastocysts for mouse generation. The obtained chimeric mouse lines were crossed to C57BL/6 lines for germline transmission. Chimeric mice that were positive for the targeted ES cells were germline-transferred to the F1 generation and bred on a C57BL/6 background. Heterozygous Brg1^{iR973Q/+} was crossed with Sm22αCre driver line to induce Brg1 R973Q point mutation in cardiomyocytes. Upon Cre expression, Lox71 and Lox66 recombined to invert their flanked sequence. As a result, a wild type LoxP site and a new mutant site Lox72 would be created, and the inverted sequences containing R973 within exon20 would be configured in the sense direction. Then the new LoxP site would recombine with 5′ distal LoxP, which disabled wild type gene expression and activated expression of the mutant version, respectively. Such site-specific Brg1 R973Q point mutation was detected by PCR and verified by sequencing (Data not shown).

### EXAMPLE 25: YEAST GENETICS AND RNA M⁶A ANALYSIS

URA gene was cloned from YIp5 [pRB12] (37061, ATCC) plasmid, and inserted into DNA templates with R988Q or H1188A/D1190A point mutations after the stop codon of SNF2 (Data not shown). The yeast wild type and IME4 knockout (ime4Δ) strains purchased from Transomic Technologies (BY4741, Cat No: TKY0002) were transformed with linearized double strand DNA templates using PEG and LiAc. The transformation mixture was directly spread onto SD-URA selection plates. Colonies on the plates were picked, and their genomic DNA extracted for genotyping. Genotyping was performed by restriction enzyme (SmlI and HindIII) digestion followed by DNA sequencing. Total RNAs was purified from the cells using acid-phenol extraction. Enrichment of polyadenylated RNAs (polyA+ RNA) from total RNAs was performed using one round of PolyATtract® mRNA Isolation Systems (Promega).

### EXAMPLE 26: STATISTICAL ANALYSIS

Statistical significances and enzyme kinetics constants were calculated using GraphPad Prism 5 and 7 (GraphPad Software, La Jolla, Calif.). A value of P<0.05 was considered statistically significant. The software of GraphPad Prism facilitates the fitting of non-linear regression model and calculation of kinetics constants. The errors and r-square (r²) were generated from the fitting curve.

### EXAMPLE 27

Previous studies revealed that the SWI/SNF-like chromatin remodeler Brg1 maintains Bmp10 mRNA levels in embryonic hearts to promote cardiomyocyte proliferation, and the regulation of Bmp10 by Brg1 doesn't require promoter activation by Brg1, given that Brg1 doesn't bind Bmp10 promoter (Data not shown). We further tested the transcriptional activity of Brg1 on Bmp10 promoter by reporter assays. We cloned Bmp10 promoter (−5307 to +1134 bp) into pREP4 episomal reporter that allows promoter chromatinization in mammalian cells. We then transfected Brg1-expressing plasmids with the reporter into SW13 cells. In these cells Brg1 showed no activity in Bmp10 promoter, while Brg1 caused 1.7-fold increase of Myh7 promoter and 40% reduction of Myh6 promoter activity (Data not shown). Reporter studies, combined with in vivo ChIP results, indicate that Brg1 doesn't activate Bmp10 promoter, in contrast to Brg1's transcriptional activities on Myh promoters. Given that Brg1 can directly bind RNA with high affinity through its helicase domain (also known as ATPase domain), we examined the helicase domain for clues of its RNA function. Brg1 helicase domain contains two RecA-like subdomains DExxC (D1) and HELICc (D2) connected by an insertion peptide (FIG. 1). The insertion peptide harbored R/R motif (RLHKVLRPFLLRR) SEQ ID NO 4, while D2 subdomain contained H/D motif (HQDLAAQDRAH) SEQ ID NO 5 (FIG. 1), similar to the catalytic motifs (RXXXXXR and HXDXnH) of RNA m⁶A demethylase FTO and ALKBH5. Given that both R/R and H/D motifs were present in SWI/SNF proteins of yeast, drosophila, zebrafish, mouse, and humans (See FIG. 1 and FIG. 20), we investigated whether R/R and H/D motifs had any roles in m⁶A demethylation.

### EXAMPLE 28

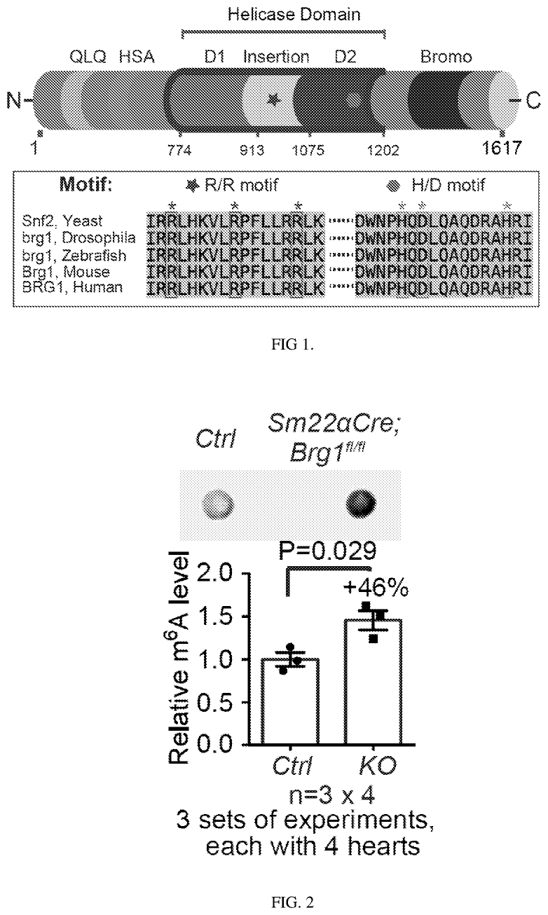

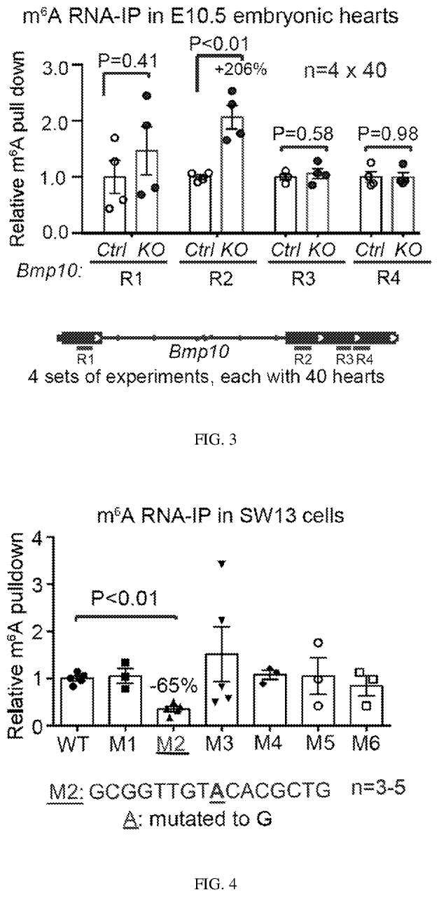

To assess whether Brg1 was an m⁶A demethylase, we began by examining m⁶A marks of polyadenylated RNAs in mouse embryonic hearts with or without Brg1 in cardiomyocytes. To delete Brg1 in fetal cardiomyocytes, we used Sm22α promoter to drive the expression of Cre recombinase in fetal cardiomyocytes to recombine Brg1 floxed alleles (Brg1^{fl/fl}) in Sm22αCre;Brg1^{fl/fl} mice, allowing the disruption of myocardial Brg1 by embryonic day 8.5-9.5 (E8.5-9.5). To quantitate m⁶A methylation, we used dot blot analysis with m⁶A-specific antibody and found that m⁶A marks of total polyadenylated RNAs of E10.5 mouse hearts increased by 46% when Brg1 was absent in cardiomyocytes (Sm22αCre;Brg1^{fl/fl}), relative to the littermate control hearts (Sm22αCre;Brg1^{fl/+}, Brg1^{fl/+} or Brg1^{fl/fl}) (See FIG. 2). This enhanced RNA m⁶A methylation was confirmed by two-dimensional thin layer chromatography (2D-TLC), which showed 44% increase of m⁶A-to-A ratio in those E10.5 Brg1-null hearts (Data not shown). Such m⁶A changes occurred in the absence of protein changes of m⁶A reader (YTHDF2), methyltransferases (METTL3 and 14), or demethylases (FTO, ALKBH5) (Data not shown). These observations are consistent with Brg1 functioning as an m⁶A demethylase in the embryonic hearts.

### EXAMPLE 29

Given that Brg1 is essential for maintaining Bmp10 mRNA levels to promote fetal cardiomyocyte proliferation, we asked whether the m⁶A status of Bmp10 mRNA was altered in embryonic hearts lacking Brg1 in cardiomyocytes. To test that, we used m⁶A-RNA immunoprecipitation coupled with RT-qPCR (m⁶A-RNA-IP) to precipitate m⁶A from E10.5 hearts and quantitate Bmp10 mRNA. In this assay, mRNA was fragmented for IP, and specific qPCR primers were used to target four different regions (R1-R4) in Bmp10 mRNA to assess m⁶A enrichment (See FIG. 3). The mature Bmp10 mRNA consists of two exons (exon1 base 1-373, exon2 base 374-1325). The R1 primer targeted exon1, and R2-R4 primers targeted the longer exon 2 (See FIG. 3). In E10.5 Brg1-null hearts (Sm22αCre;Brg1^{fl/fl}), m⁶A level was increased by 2.1-fold in the R2 region of Bmp10 mRNA (P<0.01) without significant changes in the other R regions (See FIG. 3), suggesting that R2 region is the predominant m⁶A site of action of Brg1 on Bmp10 mRNA. To further test whether Brg1 could remove m⁶A of Bmp10 in cells, we co-transfected Brg1 and Bmp10 expression plasmids into the BRG1- and BRM-null SW13 cells (human adrenal carcinoma cells) to reconstitute Brg1 in those cells and then quantitated Bmp10 m⁶A methylation. Indeed, m⁶A-RNA-IP analysis showed that Brg1 expression reduced m⁶A levels of Bmp10 by ˜50% in R2 (P<0.01) without significant m⁶A changes in the other R regions (Data not shown). Both genetics- and cell-based assays indicate that Brg1 is necessary for Bmp10 m⁶A demethylation in a specific region.

### EXAMPLE 30

To determine the in vivo Bmp10 site of m⁶A demethylation by Brg1, we profiled m⁶A methylation of polyadenylated RNAs in embryonic hearts, using m⁶A-RNA-IP and RNA sequencing. Polyadenylated RNAs were prepared from E10.5 hearts and fragmented to ˜100 nucleotides in length, followed by m⁶A-RNA-IP and RNA sequencing. Such m⁶A mRNA profiling—consistent with the dot blot and 2D-TLC analyses (See FIG. 2)—revealed an enhanced RNA m⁶A signals in embryonic hearts that lacked Brg1 in cardiomyocytes (Sm22αCre;Brg1^{fl/fl}), particularly in the 5′ untranslated regions (UTR) of RNAs (10,281 expressed genes) (P=0.003, two-tail t-test) (Data not shown). The consensus m⁶A sequence GGAUC in embryonic hearts was similar to that reported previously (Data not shown). Although many RNAs showed m⁶A modifications at 5′-UTR, there were also RNAs with internal or 3′-UTR m⁶A methylation controlled by Brg1. These RNAs included, but not limited to, Bmp10, Pcdh17, Anpep, Tnik and Kbtbd7 (Data not shown). For Bmp10, it was exon2 that showed dynamic m⁶A changes controlled by Brg1, with 4-fold increase of m⁶A in R2 (but not other regions) of Bmp10 exon 2 in Brg1-null hearts (Data not shown). The genome-wide m⁶A profiling therefore showed similar results derived from targeted Bmp10 m⁶A-RNA-IP and reporter assays (FIG. 3). In contrast to Bmp10, the long noncoding RNA Myheart (Mhrt), which is capable of binding Brg1 helicase to inhibit its chromatin engagement, showed no changes of m⁶A levels with or without Brg1 (Data not shown). The lack of m⁶A regulation of Mhrt by Brg1 in embryonic hearts is consistent with our previous findings that Brg1 represses Mhrt expression at the promoter and transcription level and that Mhrt knockout mice had no apparent developmental heart defects. Collectively, all three analyses of m⁶A methylation mapped R2 as the critical region for Brg1 to control Bmp10 m⁶A modification.

### EXAMPLE 31

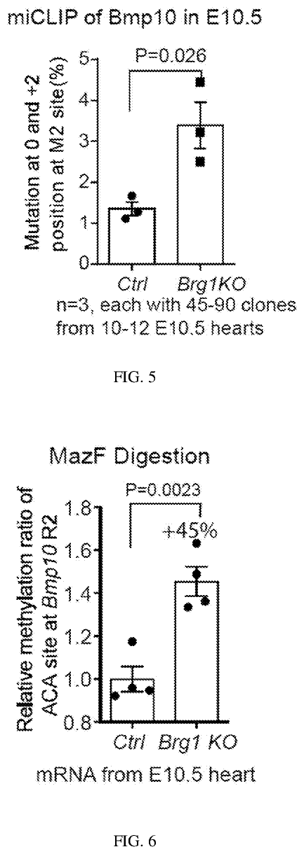

Modifications of m⁶A that control RNA stability are enriched around stop codons, in 3′UTR, or within long exons. The R2 location in the longer exon of Bmp10 fit the profile of m⁶A modifications. We then focused on R2 to determine the primary adenosine site of Bmp10 m⁶A methylation. Based on similarities to the consensus m⁶A sequence (Data not shown), we selected six potential m⁶A sites (M1-M6) from R2 for mutagenesis (A-to-G point mutation) to disable m⁶A modification (See FIG. 21). We then quantitated m⁶A changes of those Bmp10 mutants in the BRG1-null SW13 cells capable of methylating Bmp10 (Data not shown). By m⁶A-RNA-IP analysis, we found that A-to-G mutation at the M2 site (CGGUUGUACACGCUG) showing SEQ ID NO 11 within part of Region 2 SEQ ID NO 9, but not other M sites, caused 65% reduction of Bmp10 m⁶A signals (FIG. 4), suggesting that the M2 adenosine is a primary site of Bmp10 m⁶A methylation. To further determine whether the M2 adenosine was an in vivo m⁶A site controlled by Brg1, we performed m⁶A individual-nucleotide-resolution cross-linking and immunoprecipitation (miCLIP) in mouse heart tissues (Data not shown). Fragmented RNAs from E10.5 hearts were UV cross-linked to anti-m⁶A antibody and then reverse-transcribed. During reverse transcription, such crosslinking could induce mutations at position 0, +1, or +2 relative to the m⁶A site. The miCLIP analysis followed by Bmp10 PCR of E10.5 hearts showed that the M2 adenosine of Brg1-null hearts displayed significantly higher frequency of mutations at 0 (A-to-C or T) and +2 (A-to-C) positions than that of littermate control hearts (3.39±0.98% vs 1.35±0.28%)(See FIG. 5). The results indicate that the M2 adenosine is demethylated by Brg1 in embryonic hearts. The miCLIP results were further confirmed by an antibody-independent MazF enzyme digestion assay. MazF is an m⁶A-sensitive RNA endoribonuclease that specifically cleaves the unmethylated ACA sequence at the 5′-side of the first adenosine, leaving the methylated version (m⁶A)CA intact. Given the M2 sequence GUACA, the MazF method provides a precise tool to test whether the first M2 adenosine was demethylated by Brg1 in vivo. mRNAs isolated from E10.5 control and Brg1-null hearts were digested with MazF, and the digested mRNAs were subject to RT-qPCR to quantitate the cDNA spanning the M2 GUACA site. The same amount of mRNA without MazF digestion was amplified simultaneously as the input normalizer (See FIG. 22). In the Brg1-null hearts, the undigested GU(m⁶A)CA signals increased by 45% (See FIG. 6), indicating that the first M2 adenosine (GUACAC) (SEQ ID NO 10) of Bmp10 is demethylated by Brg1 in embryonic hearts.

### EXAMPLE 32

Collectively, the identification of a specific Bmp10 m⁶A site (GUACAC) (SEQ ID NO 10) demethylated by Brg1 was accomplished by multiple methods, including sequence walking, m⁶A-RNA-IP, point mutagenesis, miCLIP, and MazF studies. Furthermore, we tested whether Brg1 was required for the demethylation of m⁶Aₘ at the penultimate base of RNAs. 2D-TLC analyses showed no changes of the ratio between m⁶Aₘ and Aₘ either by BRG1 knockdown (84% efficiency) in 293T cells or E10.5 Brg1-null hearts (Data not shown). These observations suggest a primary function of Brg1 in m⁶A but not m⁶Aₘ demethylation.

### EXAMPLE 33

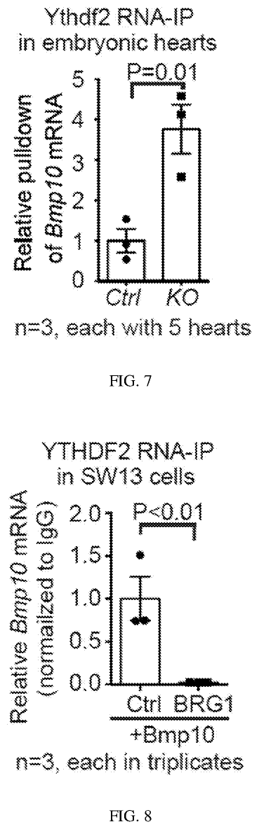

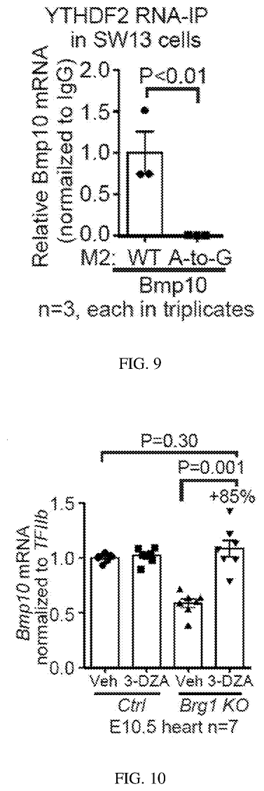

We then asked whether m⁶A demethylation of Bmp10 by Brg1 was relevant to Bmp10 stability in vivo. Given that m⁶A is selectively bound by YTH domain family 2 protein (YTHDF2) to trigger mRNA degradation, we examined the binding of endogenous Ythdf2 proteins to Bmp10 mRNA in mouse embryonic hearts with or without Brg1. RNA IP using anti-Ythdf2 antibody showed that the binding of Ythdf2 to Bmp10 mRNA was increased by 3.8-fold in E10.5 Brg1-null hearts (Sm22αCre;Brg1^{fl/fl})(See FIG. 7). This indicates that Brg1 prevents Ythdf2-Bmp10 binding to protect Bmp10 from degradation in embryonic hearts. We then tested whether Brg1 inhibited Ythdf2's binding through the m⁶A (M2) site of Bmp10. By YTHDF2-RNA-IP, we found that endogenous YTHDF2 proteins in BRG1/BRM-null SW13 cells were capable of binding transfected Bmp10 mRNA (FIG. 8), and such YTHDF2-Bmp10 binding was abolished when Brg1 was expressed in those SW13 cells (FIG. 8), recapitulating in vivo observations in embryonic hearts (See FIG. 7). We next asked whether the M2 adenosine was required for YTHDF2-Bmp10 binding. Indeed, A-to-G mutation at the M2 adenosine abolished the ability of YTHDF2 to bind Bmp10 in SW13 cells (FIG. 9). This finding indicates that YTHDF2 recognizes Bmp10 through m⁶A methylation of the M2 adenosine, which was controlled by Brg1 both in cells and in embryonic hearts. Therefore, Brg1 and YTHDF2 counteract each other's function through the same m⁶A site of Bmp10 mRNA.

### EXAMPLE 34

We further confirmed the interaction between Brg1 and YTHDF2 in controlling Bmp10 mRNA stability in cardiomyocytes. We first cultured cardiomyocytes isolated from E18.5 mouse hearts and measured Bmp10 m⁶A methylation and stability. Brg1 knockdown by siRNA in cardiomyocytes increased Bmp10 m⁶A signal at R2 (but not other R regions) by 50%, and this was associated with 70% Bmp10 mRNA reduction and 45% Bmp10 protein reduction, as well as a switch of Myh isoform expression (Myh7-to-Myh6 switch) (Data not shown). These results from cultured cardiomyocytes recapitulated the in vivo effects of Brg1 in embryonic hearts on Bmp10 and Myh expression and on Bmp10 m⁶A methylation (See FIG. 2), thus validating this approach. We then measured the stability of endogenous Bmp10 mRNA in mouse cardiomyocytes with or without Brg1 or Ythdf2 knockdown (Data not shown). Pulse-chase experiments with 4-thiouracil (4tU) were conducted to label newly synthesized Bmp10 mRNA and track its degradation in E18.5 mouse cardiomyocytes. Within four hours of chasing, the control cardiomyocytes had 4tU-labled Bmp10 reduced by 49%, while Brg1-siRNA-treated cardiomyocytes had 75% reduction of labeled Bmp10 (Data not shown). Therefore, in the absence of Brg1, Bmp10 degradation increased by 53% (P=0.036). To test the role of Ythdf2 in Bmp10 degradation, we knocked down both Brg1 and Ythdf2 to conduct Bmp10 rescue experiments. Remarkably, Bmp10 degradation caused by Brg1 knockdown was fully rescued by Ythdf2 knockdown (Data not shown). These findings were further confirmed by studies using actinomycin D to inhibit transcription and halt new mRNA production in cardiomyocytes (Data not shown). Therefore, in cardiomyocytes Brg1 stabilizes Bmp10 mRNA by inhibiting Ythdf2-Bmp10 binding to prevent RNA degradation.

### EXAMPLE 35

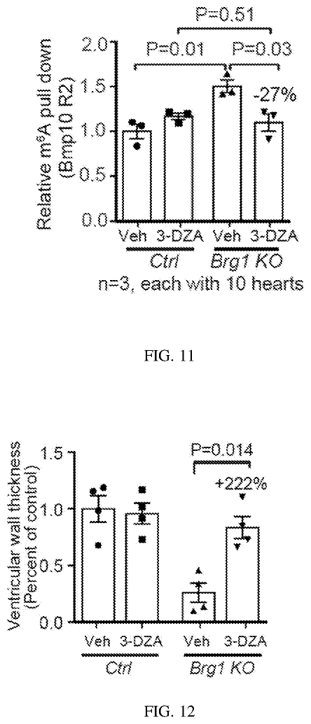

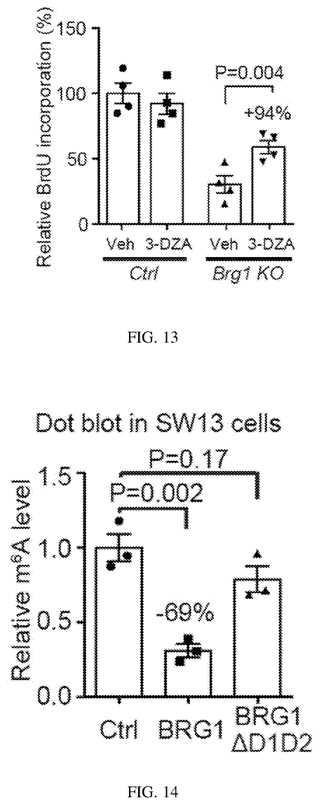

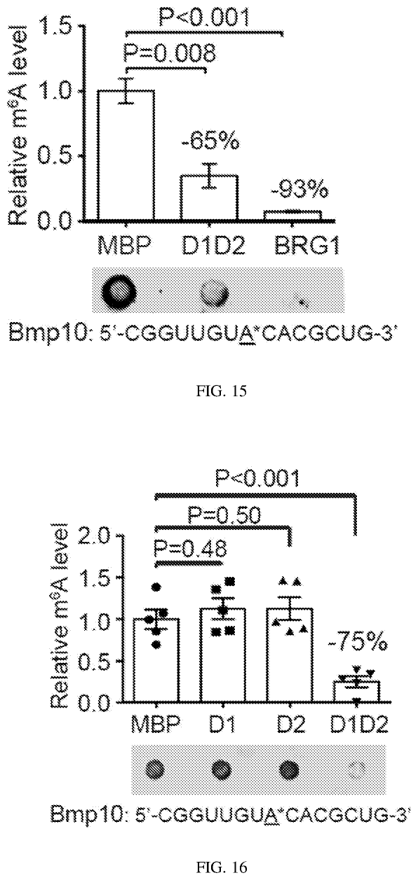

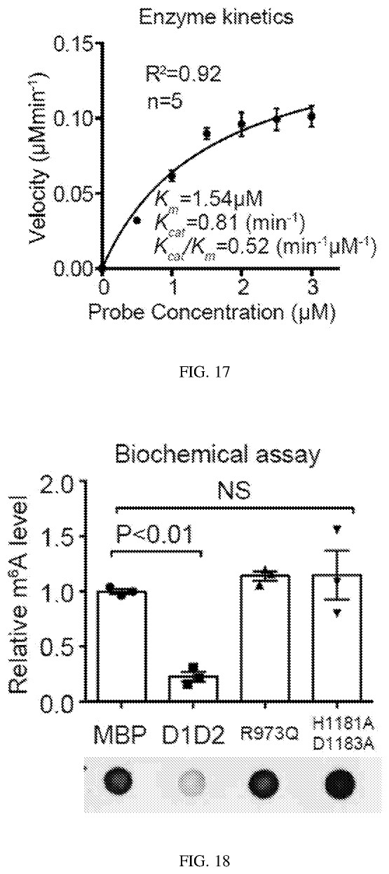

Given the ability of Brg1 to erase m⁶A marks from Bmp10 (See FIGS. 3, 4, and 5) and to prevent Bmp10 degradation in cardiomyocytes (Data not shown), we asked whether such Brg1-mediated Bmp10 m⁶A demethylation was essential for in vivo fetal cardiomyocyte proliferation. To test that, we used 3-deazaadenosine (3-DZA) to inhibit m⁶A methylation and conducted rescue experiments of cardiomyocyte proliferation and ventricular wall formation in Brg1-null hearts. We reasoned that 3-DZA, by inhibiting methylation, should counteract the ectopic Bmp10 m⁶A hypermethylation and degradation in Brg1-null hearts. 3-DZA or the control vehicle was injected into pregnant mice starting from gestational period E7.5 to E10.5, and the hearts of embryos were harvested at E10.5 for analyses. In control littermate hearts (Sm22αCre;Brg1^{fl/+}, Brg1^{fl/+} or Brg1^{fl/fl}) that had abundant Brg1 in cardiomyocytes, 3-DZA did not alter Bmp10 mRNA and protein levels (See FIG. 10). Conversely, in Brg1-null hearts (Sm22αCre;Brg1^{fl/fl}), Bmp10 mRNA and Bmp10 proteins were reduced by 40% and 57% respectively, and 3-DZA enhanced Bmp10 mRNA and Bmp10 protein levels and normalized Bmp10 proteins in those hearts (See FIG. 10). Concurrent with BMP10 mRNA and protein normalization, 3-DZA reduced Bmp10 m⁶A methylation and normalized Bmp10 m⁶A levels at the R2 site in Brg1-null hearts (FIG. 11). Such 3-DZA-mediated reduction of Bmp10 m⁶A and increase of Bmp10 was associated with a 2.2-fold increase of myocardial wall thickness (See FIG. 12) and 94% increase and normalization of cardiomyocyte proliferation measured by BrdU incorporation (See FIG. 13). The increase of myocyte proliferation in Brg1-null hearts treated with 3-DZA was confirmed by double immunostaining and colocalization of phospho-Histone 3 and cardiac Troponin T, which are markers of cell mitosis and cardiomyocytes, respectively (Data not shown). Remarkably, 3-DZA, despite its profound effect on cardiomyocyte proliferation, showed no significant effects on Myh isoform switch in Brg1-null hearts (FData not shown). The differential rescue of myocyte proliferation (but not Myh switch) by 3-DZA was identical to the rescuing effect of Bmp10 proteins on Brg1-null hearts, indicating that Bmp10 m⁶A demethylation by Brg1 is essential for maintaining Bmp10 protein level for heart muscle development in vivo.

### EXAMPLE 36

To further pursue this new molecular action of Brg1, we tested whether Brg1 had a general role in RNA m⁶A demethylation in other cells beyond cardiomyocytes. We used siRNA to knock down BRG1 in human 293T cells that do not express Bmp10 and obtained 84% efficiency of BRG1 disruption without affecting the expression of m⁶A reader (YTHDF2), methyltransferases (METTL3 and 14), or demethylases (FTO, ALKBH5) (Data not shown). Quantitation of m⁶A of mRNAs showed that BRG1 knockdown caused 64% increase of m⁶A (dot blot) and 58% increase of m⁶A-to-A ratio (2D-TLC) on polyadenylated RNAs (Data not shown). The increase of m⁶A was further confirmed by liquid chromatography-tandem mass spectrometry (LC-MS/MS), which showed a 48% increase of m⁶A/A signal intensity ratio when BRG1 was knocked down by 65% (Data not shown). Therefore, without changing the expression of known m⁶A reader, writer, or eraser, Brg1 is necessary for RNA m⁶A demethylation in 293T cells. Therefore, Brg1 is capable of erasing m⁶A marks from RNAs in multiple cell types.

### EXAMPLE 37

Given that Brg1 helicase domain carried putative motifs of an m⁶A demethylase (See FIG. 1), we asked whether the helicase domain was essential for Brg1 to erase RNA m⁶A marks. In BRG1 knockdown 293T cells or BRG1/BRM-null SW13 cells, transfection of BRG1 caused reduction of RNA m⁶A levels, whereas transfection of Brg1 that lacked helicase domain (Brg1-ΔD1D2) had no significant impact on m⁶A levels, despite the robust expression of Brg1-ΔD1D2 proteins (See FIG. 14). Then, we examined cell-by-cell the correlation between the presence of Brg1 proteins and m⁶A marks by co-immunostaining with anti-Brg1 and anti-m⁶A antibodies. Co-immunostaining showed that the control human SW13 cells had no Brg1 proteins and contained abundant m⁶A marks, predominantly in the cytosol with a few speckles in the nucleus (Data not shown). Remarkably, in cell cultures transfected with wildtype Brg1, those cells that incorporated and expressed Brg1 lost 66% of m⁶A marks, whereas cells that didn't uptake Brg1 retained m⁶A in both the nucleus and cytosol (Data not shown). Conversely, cells transfected with helicase-null Brg1 (Brg1-ΔD1D2) maintained their m⁶A marks in a pattern resembling that of control SW13 cells (Data not shown). These immunostaining results revealed the necessity of Brg1 and its helicase domain for m⁶A demethylation. Such helicase-dependent m⁶A demethylation was further confirmed by reconstitution of Brg1 or Brg1-ΔD1D2 in human lung carcinoma H1299 cells that lack BRG1²⁵ (Data not shown). The exclusive nuclear localization of Brg1 proteins (Data not shown) suggests that m⁶A demethylation by Brg1 takes place before RNA transcripts exit the nucleus, consistent with the observation that m⁶A methylation and demethylation events occur primarily in the nucleus. Taken together, these results indicate that Brg1, through its helicase (also referred to herein as its D1D2) domain, is capable of erasing m⁶A marks in cells.

### EXAMPLE 38

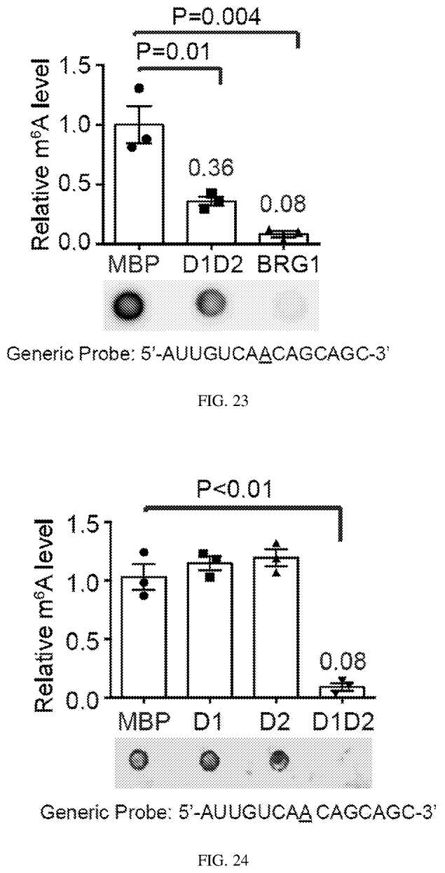

Next we tested whether Brg1 was in itself an m⁶A demethylase. We used recombinant proteins and synthetic RNA probes to examine the biochemical activity of Brg1 as an RNA m⁶A demethylase. We purified maltose-binding-protein (MBP)-tagged Brg1 helicase domain proteins (D1D2, amino acid 774-1202) (Data not shown) and synthesized an m⁶A-containing Bmp10 mRNA probe (5′-CGGUUGUA*CACGCUG-3′, A*: m⁶A) SEQ ID NO 12 with m⁶A at the M2 adenosine site (See FIGS. 23 and 24). The m⁶A-Bmp10 RNA probe was incubated with Brg1 proteins and quantitated for m⁶A by dot blot analysis. We found that both full-length Brg1 proteins and the helicase domain (MBP-D1D2) (SEQ ID NO 27 coupled to SEQ ID NO 2) effectively erased the m⁶A mark from Bmp10 (FIG. 15), whereas the subdomain D1 (amino acid 774-913) (SEQ ID NO 6) or D2 (amino acid 1075-1202) (SEQ ID NO 7) alone was incapable of doing that (FIG. 16). To exclude RNA degradation, we verified the integrity of Bmp10 RNA after demethylation reactions (Data not shown). We further verified the removal of N⁶-methyl group from the M2 adenosine by LC-MS/MS. Bmp10 RNA probe with m⁶A-modified M2 site was incubated with MBP and MBP-D1D2 recombinant proteins, followed by enzyme digestions to single nucleosides and LC-MS/MS analyses. The adenosine and m⁶A signals were verified by pentose removal (Data not shown). Based on A and m⁶A signal intensity, there was a 40% reduction of m⁶A/A ratio upon Brg1 D1D2 treatment (Data not shown). The LC-MS/MS results corroborated findings from m⁶A dot blot and 2D-TLC analyses, revealing a direct m⁶A-demethylating activity of Brg1. Furthermore, by electrophoretic mobility shift assay, we found that the helicase D1D2 preferentially bound to m⁶A-containing Bmp10 probe, but not the unmethylated version (Data not shown), with a strong binding affinity (Kd=0.176 +/−0.064 μM) (Data not shown). Bmp10 m⁶A demethylation by Brg1 helicase displayed the following kinetics constants: Km (Michaelis constant) 1.54 μM, Kcat (catalytic rate) 0.81 min⁻¹, and Kcat/Km ratio (catalytic efficiency) 0.52 min⁻¹ μM⁻¹ (See FIG. 17). The enzyme kinetics of Brg1 was similar to that of the FTO m⁶A demethylase (Km=0.41 μM, Kcat=0.30 min⁻¹, Kcat/Km=0.72 min⁻¹ μM⁻¹), whereas Brg1 exhibited more efficient enzymatic activity than that of ALKBH5 (Km=1.38 μM, Kcat=0.17 min⁻¹, Kcat/Km=0.12 min⁻¹ μM⁻¹). Consistent with Brg1's general demethylation role in multiple cell types, the m⁶A demethylase activity of Brg1 was not restricted to Bmp10. Brg1 and its helicase domain were capable of removing the m⁶A mark from a generic RNA probe (5′-AUUGUCAA*CAGCAGC-3′, A*: m⁶A) (SEQ ID NO 12) used previously (See FIGS. 23 and 24). Collectively, the results demonstrate that Brg1 is an m⁶A demethylase, with the helicase domain as the catalytic unit.

### EXAMPLE 39

We then asked whether the R/R and H/D motifs of Brg1 helicase were required for Brg1's m⁶A demethylation activity. We mutated R973 of Brg1 R/R motif (RLHKVLR*PFLLRR, R*: R973) (modified SEQ ID NO 4) to Q973 (FIG. 1 and FIG. 20) and quantitated the helicase's activity on the m⁶A-Bmp10 probe. R973Q mutation abolished the catalytic activity of Brg1 helicase: D1D2-R973Q helicase was unable to demethylate m⁶A-Bmp10 in dot blot analysis (FIG. 18). We also tested the requirement of Brg1 H/D motif (H*QD*LAAQDRAH, H*: H1181, D*: D1183) (modified SEQ ID NO: 5) by generating H1181A and D1183A mutations (FIG. 1 and FIG. 20). Such H/D motif mutations also disabled the helicase for m⁶A demethylation (FIG. 18), demonstrating the biochemical requirement of R/R and H/D motifs for Brg1 helicase to demethylate m⁶A.

### EXAMPLE 40

To evaluate the in vivo necessity of these motifs, we generated similar R/R and H/D mutations in the context of full-length Brg1 and tested their m⁶A activities in BRG1 knockdown 293T cells and BRG1/BRM-null SW13 cells. Wildtype and mutant Brg1 constructs (Brg1, Brg1-R973Q, or Brg1-H1181A/D1183A) were respectively transfected to both types of cells to reconstitute Brg1 proteins (Data not shown). Dot blot m⁶A quantitation showed that Brg1-R973Q and Brg1-H1181A/D1183A mutant proteins lost their ability to demethylate m⁶A in cells (Data not shown). The loss of m⁶A demethylation by Brg1 mutant proteins was further confirmed by cellular co-immunostaining of m⁶A and Brg1 proteins in SW13 cells transfected with Brg1 mutant constructs. Cells that incorporated Brg1-R973Q or Brg1-H1181A/D1183A proteins maintained their m⁶A marks comparable to control cells (Data not shown), corroborating the results of biochemical studies (Data not shown). Next, we tested whether ATP hydrolysis by Brg1 was essential for m⁶A demethylation. Cells reconstituted with Brg1-K798R that had ATPase disabled (Data not shown) showed ˜50% reduction of m⁶A signal, comparable to that achieved by wildtype Brg1 (Data not shown). Conversely, R973Q and H1181A/D1183A proteins, though incapable of m⁶A demethylation, maintained their biochemical ability to hydrolyze ATP (Data not shown), suggesting a separation of ATPase and m⁶A demethylation function of Brg1. Furthermore, to test whether R/R or H/D mutations affected chromatin remodeling, we used histone octamers to chromatinize in vitro the Myh6 promoter DNA (−428 to −227 upstream of Myh6 TSS), which Brg1 is capable of binding, remodeling, and transcriptionally repressing. We found that nucleosomes assembled on the Myh6 promoter were remodeled by Brg1 R973Q and H1181A/D1183A proteins, as evidenced by the removal of histones from Myh6 DNA, releasing the free DNA (Data not shown). Therefore, R973 and H1181/D1183 are not essential for chromatin remodeling. Collectively, these biochemical and cell-based studies identify the catalytic motifs and establish Brg1 as an RNA m⁶A demethylase, independent of its ATPase and chromatin remodeling activity.

### EXAMPLE 41

R973 in R/R motif played a dominant role in m⁶A demethylation, and remarkably, heterozygous R973 missense mutations (R973Q and R973W) were frequently found in human cancers. Such R973 mutations, however, had no known effects on Brg1's chromatin engagement or ATP hydrolysis, consistent with our biochemical studies (Data not shown). Given its implications in disease biology, we tested whether R973 was essential for Brg1's m⁶A demethylase activity in vivo by generating a murine germline R973Q (CGG-to-CAG) mutation. To our surprise, despite multiple attempts, we were unable to obtain any heterozygous line viable to birth, suggesting embryonic lethality of R973Q heterozygosity. To overcome that, we designed a genetic knock-in scheme to enable inducible, tissue-specific R973Q mutation (Data not shown). By embryonic stem cell gene targeting, we introduced into Brg1 locus an inversion cassette, flanked by LoxP and Lox71/Lox66 sites and comprising duplicated exon20 of Brg1—a wild type exon20 and an inverted one with R973Q (Data not shown). In the absence of Cre recombinase, the cassette allowed wildtype exon20 to express; conversely, with Cre activity the cassette was inverted and switched the expression to R973Q-exon20. We then used Sm22αCre to induce cardiomyocyte-specific Brg1-R973Q mutation (Data not shown). Even with such tissue-specific R973Q mutation, we encountered heterozygous lethality between E14.5 to birth in embryos (Data not shown). In E10.5-12.5 R973Q heterozygous hearts (Sm22αCre;Brg1^{iR973Q/+}), dot blot analysis showed that m⁶A-modified mRNAs increased by 115% (Data not shown), and miCLIP analyses of E12.5 R973Q hearts revealed that Bmp10 mutations occurred at 0 (A-to-C/T) and +2 (A-to-C) positions relative to the M2 adenosine with significantly higher frequencies than the littermate control hearts (3.6±0.88% vs 1.3±1.1%)(Data not shown). These results indicate that R973 is essential for Brg1 to demethylate the M2 adenosine of Bmp10 in embryonic hearts. In those R973Q heterozygous hearts, Brg1 protein levels were comparable to controls, while Bmp10 mRNA and protein levels decreased by 30% (Data not shown). The changes of Bmp10 m⁶A and mRNA were associated with a 45% decrease of myocardial wall thickness at E13.5 (Data not shown). The in vivo genetic studies thus demonstrate that R973 is essential for Brg1-mediated m⁶A demethylation necessary for heart muscle development.

### EXAMPLE 42

Given that Brg1 represents the vertebrate SWI/SNF family of ATP-dependent chromatin-remodeling factors, we asked whether the m⁶A-demethylating activity of Brg1 was conserved in its yeast orthologue Snf2 of Saccharomyces cerevisiae. The yeast contains methyltransferase complex (MIS), composed of Ime4 (METTL3 orthologue), Mum2 (WTAP orthologue), and Slz1. Mutations of the MIS proteins abrogate m⁶A methylation of mRNAs and delay meiotic entry. Despite the biological importance of m⁶A methylation, enzymes that demethylate m⁶A in yeasts have not been identified.

### EXAMPLE 43

We found that the yeast chromatin remodeler Snf2 also contained R/R and H/D motifs identical to those of mouse Brg1 (See FIG. 1), suggesting that Snf2 was an m⁶A demethylase. To test that, we generated MBP-tagged Snf2 helicase domain (amino acid 787-1211) (Data not shown) and examined its biochemical ability as an m⁶A demethylase. This Snf2 helicase domain was indeed biochemically capable of demethylating m⁶A-Bmp10 RNA probe (Data not shown). Next, we tested the in vivo activity of Snf2 as an m⁶A demethylase. Although controversial, a recent study suggested the presence of m⁶A-modified RNAs in yeast haploid cells. To further define m⁶A regulation in yeast, we used multiple methods (dot blot, 2D-TLC, and LC-MS/MS) to demonstrate RNA m⁶A signals in yeast haploid cells and a reduction of m⁶A signal in cells that lacked Ime4 methyltransferase (ime4Δ) (Data not shown). The m⁶A signals detected by multiple independent methods and the requirement of an m⁶A methyltransferase strongly support the presence of m⁶A regulation in yeast haploid cells. We then generated in haploid cells point mutations of SNF2 in R/R and H/D motifs: snf2R988Q and snf2H1188A, D1190A (Data not shown). Yeasts that carried snf2R/R or H/D motif point mutations exhibited growth impairment and produced smaller colonies, and complete SNF2 knockout (snf2Δ) caused the most severe growth retardation (Data not shown). Dot blot m⁶A analysis of polyadenylated RNAs isolated from yeasts of snf2Δ, snf2R988Q, or snf2H1188A, D1190A mutations showed >100% enhancement of RNA m⁶A methylation (Data not shown), which was further confirmed by 2D-TLC and LC-MS/MS (Data not shown). The biochemical and genetics studies collectively demonstrate the role of Snf2 as an m⁶A demethylase in yeast.

### EXAMPLE 44

To test whether Snf2 counteracted Ime4 in RNA m⁶A regulation, we generated yeast haploid strains with SNF2 and IME4 double mutations—ime4Δsnf2R988Q and ime4Δsnf2H1188A, D1190A. Remarkably, the snf2R/R or H/D point mutation fully rescued the reduction of global RNA m⁶A signal in ime4Δ cells (Data not shown). Such opposing regulation of m⁶A by Ime4 and Snf2 at the global level strongly supports the role of Snf2 as an m⁶A demethylase. However, unlike SNF2 mutants, the ime4Δ haploid cells produced colonies with comparable size to that of the parent wildtype strain, indicating that Ime4 isn't essential for regulating specific RNA(s) required for haploid cell growth. Consistent with this non-essentiality for cell growth, ime4Δ couldn't rescue the growth defects of SNF2 mutants (snf2R988Q and snf2H1188A, D1190A)(Data not shown). The results suggest that these two m⁶A regulators, despite having common RNA targets globally, show distinct, non-overlapping subsets of downstream RNAs that await further characterization.

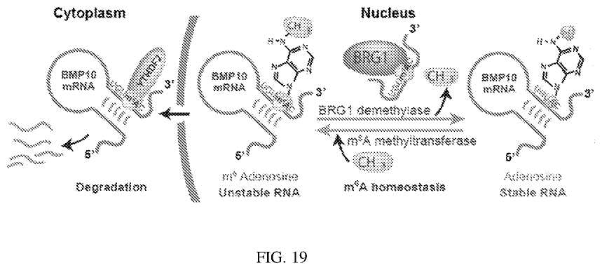

These studies describe a new class of RNA m⁶A demethylase evolutionarily conserved from yeast to mammals. The Brg1-mediated RNA m⁶A regulation not only depicts a new mechanistic terrain for heart development, but also glimpses a wider vista of the molecular action of SWI/SNF proteins beyond their traditional role as chromatin regulators (See FIG. 19). Our findings have major mechanistic implications in heart muscle development, chromatin-gene regulation, cancer biology, and eukaryotic RNA regulation. In mouse embryos, Brg1 assembles a chromatin-modifying complex with HDAC and PARP on Myh promoters to control Myh isoform transcription and cardiomyocyte differentiation. In parallel, Brg1 enzymatically removes Bmp10 m⁶A marks at a specific adenosine site to prevent Bmp10 mRNA degradation to promote cardiomyocyte proliferation. Therefore, Brg1 functions through distinct mechanisms (chromatin-transcription and RNA stability regulation) to orchestrate cardiomyocyte differentiation and proliferation, setting a stage for future studies to identify new chromatin-m⁶A modifiers and elucidate m⁶A-based mechanisms of heart muscle development. The m⁶A-based regulation also provides a mechanism to fine-tune Bmp10 levels and heart muscle growth, suggesting a therapeutic strategy for growing cardiomyocytes.

### EXAMPLE 45

RNA m⁶A modification adds to the diversity of Brg1 function in chromatin remodeling, transcription control, and RNA regulation (Data not shown). To execute those functions, the Brg1 helicase domain mediates ATP hydrolysis, chromatin engagement, and RNA binding and m⁶A demethylation. Brg1 has at least two different modes of direct interaction with RNA molecules. Binding to the lncRNA Mhrt allows Brg1 to modulate its chromatin binding and gene targeting, whereas binding to m⁶A-modified Bmp10 or other RNAs enables Brg1 to maintain RNA stability after transcription. Besides stabilizing RNAs through exonic/3′-UTR m⁶A demethylation, Brg1 may act on 5′-UTR m⁶A to regulate cap-independent RNA translation, given the enhanced m⁶A signals at 5′-UTR in Brg1-null hearts. Brg1 helicase is structurally distinct from FTO and ALKBH5, and further studies will be needed to determine how Brg1's m⁶A activity is structurally supported and how Brg1 helicase physically switches between different functional modes to execute a fine control of gene expression at different levels to cope with pathophysiological demands

Brg1 is thought to act as an ATPase subunit of the multi-protein BAF chromatin-remodeling complex, and its function outside BAF is essentially unknown. As a tumor suppressor, Brg1 often has its R973 in the ATPase/helicase domain mutated to Q or W in human cancers. Those mutations, unlike other cancer-related mutations in the helicase domain, have no effects on ATP hydrolysis or nucleosome-remodeling activity of Brg1 (current study). Accordingly, the inability of R973Q mutation to demethylate m⁶A reveals a new m⁶A-based mechanism of Brg1 in tumor suppression. Brg1 may m⁶A-demethylate a subset of tumor suppressor genes to stabilize their mRNAs and inhibit cancer progression. Consistent with this model, the m⁶A methyltransferase METTL3 was shown to repress a tumor suppressor gene SOCS2 through an m⁶A-dependent mechanism in hepatocellular carcinoma to promote cancer progression. Identification of m⁶A targets of Brg1 in cancer cells may be useful to fully evaluate the m⁶A mechanism of Brg1 in cancer biology.

### EXAMPLE 46

The m⁶A action of SWI/SNF chromatin remodelers delivers the first example of yeast RNA m⁶A demethylase (Swi2/Snf2), providing a major missing component in the eukaryotic m⁶A regulatory circuit. In yeast, m⁶A-modified RNAs are thought to accumulate only during meiosis and absent in haploid cells. By multiple independent chemical methods, we demonstrated the presence of m⁶A-modified mRNAs in haploid cells and their antithetical m⁶A regulations by the Ime4 methyltransferase and Snf2 demethylase, extending the m⁶A biology beyond meiosis. The findings introduce an extra layer of gene regulation by SWI/SNF proteins besides their chromatin-based gene transcription control. Future studies are essential to decipher how the SWI/SNF complex may couple m⁶A regulation to its chromatin and transcription function and how the four helicase-defined families of ATP-dependent chromatin remodelers differ in this new aspect of their biology. Given these unexpected molecular actions of SWI/SNF proteins, gene changes caused by chromatin remodelers will need to be re-interpreted and understood in the context of chromatin regulation and post-transcriptional m⁶A modification.

SEQUENCE LISTING

SEQ ID NO 1: Brg1-Mouse
MSTPDPPLGGTPRPGPSPGPGPSPGAMLGPSPGPSPGSAHSMMGPSPGPPSAGHPMPTQ
GPGGYPQDNMHQMHKPMESMHEKGMPDDPRYNQMKGMGMRSGAHTGMAPPPSPM
DQHSQGYPSPLGGSEHASSPVPASGPSSGPQMSSGPGGAPLDGSDPQALGQQNRGPTPF
GPGPGPGPGPGPGPGPAPPNYSRPHGMGGPNMPPPGPSGVPPGMPGQPPGGPPKPWPE
GPMANAAAPTSTPQKLIPPQPTGRPSPAPPAVPPAASPVMPPQTQSPGQPAQPAPLVPLH
QKQSRITPIQKPRGLDPVEILQEREYRLQARIAHRIQELENLPGSLAGDLRTKATIELKAL
RLLNFQRQLRQEVVVCMRRDTALETALNAKAYKRSKRQSLREARITEKLEKQQKIEQE
RKRRQKHQEYLNSILQHAKDFREYHRSVTGKLQKLTKAVATYHANTEREQKKENERI
EKERMRRLMAEDEEGYRKLIDQKKDKRLAYLLQQTDEYVANLTELVRQHKAAQVAK
EKKKKKKKKKAENAEGQTPAIGPDGEPLDETSQMSDLPVKVIHVESGKILTGTDAPKA
GQLEAWLEMNPGYEVAPRSDSEESGSEEEEEEEEEEQPQPAQPPTLPVEEKKKIPDPDS
DDVSEVDARHIIENAKQDVDDEYGVSQALARGLOSYYAVAHAVTERVDKQSALMVN
GVLKQYQIKGLEWLVSLYNNNLNGILADEMGLGKTIQTIALITYLMEHKRINGPFLIIVP
LSTLSNWAYEFDKWAPSVVKVSYKGSPAARRAFVPQLRSGKFNVLLTTYEYIIKDKHIL
AKIRWKYMIVDEGHRMKNHHCKLTQVLNTHYVAPRRLLLTGTPLQNKLPELWALLNF
LLPTIFKSCSTFEQWFNAPFAMTGEKVDLNEEETILIIRRLHKVLRPFLLRRLKKEVEAQ
LPEKVEYVIKCDMSALQRVLYRHMQAKGVLLTDGSEKDKKGKGGTKTLMNTIMQLR
KICNHPYMFQHIEESFSEHLGFTGGIVQGLDLYRASGKFELLDRILPKLRATNHKVLLFC
QMTSLMTIMEDYFAYRGFKYLRLDGTTKAEDRGMLLKTFNEPGSEYFIFLLSTRAGGL
GLNLQSADTVIIFDSDWNPHQDLQAQDRAHRIGQQNEVRVLRLCTVNSVEEKILAAAK
YKLNVDQKVIQAGMFDQKSSSHERRAFLQAILEHEEQDEEEDEVPDDETVNQMIARHE
EEFDLFMRMDLDRRREEARNPKRKPRLMEEDELPSWIIKDDAEVERLTCEEEEEKMFG
RGSRHRKEVDYSDSLTEKQWLKTLKAIEEGTLEEIEEEVRQKKSSRKRKRDSEAGSSTP
TTSTRSRDKDEESKKQKKRGRPPAEKLSPNPPNLTKKMKKIVDAVIKYKDSSSGRQLSE
VFIQLPSRKELPEYYELIRKPVDFKKIKERIRNHKYRSLNDLEKDVMLLCQNAQTFNLE
GSLIYEDSIVLQSVFTSVRQKIEKEDDSEGEESEEEEEGEEEGSESESRSVKVKIKLGRKE
KAQDRLKGGRRRPSRGSRAKPVVSDDDSEEEQEEDRSGSGSEED
SEQ ID NO 2: D1D2 (mouse) amino acid
NGILADEMGLGKTIQTIALITYLMEHKRINGPFLIIVPLSTLSNWAYEFDKWAPSVVKVS
YKGSPAARRAFVPQLRSGKFNVLLTTYEYIIKDKHILAKIRWKYMIVDEGHRMKNHHC
KLTQVLNTHYVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDMSALQRVLYR
HMQAKGVLLTDGSEKDKKGKGGTKTLMNTIMQLRKICNHPYMFQHIEESFSEHLGFT
GGIVQGLDLYRASGKFELLDRILPKLRATNHKVLLFCQMTSLMTIMEDYFAYRGFKYL
RLDGTTKAEDRGMLLKTFNEPGSEYFIFLLSTRAGGLGLNLQSADTVIIFDSDWNPHQD
LQAQDRAHRIGQQNEVRVLRL
SEQ ID NO 3: R/R + H/D Motif amino acid
IRRLHKVLRPFLLRRLKKEVEAQLPEKVEYVIKCDMSALQRVLYRHMQAKGVLLTDGS
EKDKKGKGGTKTLMNTIMQLRKICNHPYMFQHIEESFSEHLGFTGGIVQGLDLYRASG
KFELLDRILPKLRATNHKVLLFCQMTSLMTIMEDYFAYRGFKYLRLDGTTKAEDRGML
LKTFNEPGSEYFIFLLSTRAGGLGLNLQSADTVIIFDSDWNPHQDLQAQDRAHRI
SEQ ID NO 4: R/R Motif : (mouse) amino acid
IRRLHKVLRPFLLRRLK
SEQ ID NO 5: H/D Motif: (mouse) amino acid
DWNPHQDLQAQDRAHRI
SEQ ID NO 6: D1 (MOUSE) amino acid
NGILADEMGLGKTIQTIALITYLMEHKRINGPFLIIVPLSTLSNWAYEFDKWAPSVVKVS
YKGSPAARRAFVPQLRSGKFNVLLTTYEYIIKDKHILAKIRWKYMIVDEGHRMKNHHC
KLTQVLNTHYVAPRRLLLTGTPL
SEQ ID NO 7: D2 (MOUSE) amino acid
NHKVLLFCQMTSLMTIMEDYFAYRGFKYLRLDGTTKAEDRGMLLKTFNEPGSEYFIFL
LSTRAGGLGLNLQSADTVIIFDSDWNPHQDLQAQDRAHRIGQQNEVRVLRL
SEQ ID NO 8: Bmp10 DNA (mouse)
CTAGGTTGGCCTGGGAGCTGAGCAGAGAGTCATGGGGTCTCTGGTTCTGCCGCTGA
GCGCCGTCTTCTGCCTGGTGGCTCACTCGGCTTCTGGCAGCCCCATTATGGGCCTTG
AGCAGTCGCCCCTGGAAGAAGACATGCCCTTCTTTGATGATATCTTCACGGAGCAA
GATGGTATTGACTTCAACACACTGCTGCAGAGCATGAAGAACGAGTTTCTCAAGAC
GCTGAACTTGTCGGACATTCCTGTGCAGGACACGGGCAGAGTGGATCCACCAGAGT
ACATGCTGGAGCTCTACAACAAATTCGCCACAGACCGGACCTCCATGCCGTCTGCT
AACATCATCCGGAGCTTCAAGAACGAAGATCTGTTTTCTCAACCAGTCACTTTTAAT
GGGCTCCGGAAATATCCTCTCCTCTTCAATGTGTCTATCCCTCACCATGAAGAGGTC
GTCATGGCTGAACTGCGGTTGTACACGCTGGTGCAGAGAGATCGCATGATGTATGA
TGGCGTGGACCGTAAAATTACCATTTTTGAGGTACTAGAGAGTGCAGACGGTAGCG
AGGAGGAGAGGAGCATGCTGGTCTTAGTATCAACAGAGATCTACGGAACCAACAG
TGAGTGGGAGACATTTGACGTCACAGATGCCACCAGACGTTGGCAAAAGTCAGGC
CCATCAACCCATCAGCTGGAGATCCACATAGAAAGCAGACAAAACCAAGCTGAGG
ACACCGGAAGGGGACAACTGGAAATAGATATGAGTGCCCAAAATAAGCATGACCC
TTTGCTGGTTGTGTTTTCTGATGACCAAAGCAATGACAAGGAGCAGAAAGAAGAAC
TGAACGAATTGATCACCCATGAGCAGGATCTGGACCTGGACTCAGATGCTTTCTTC
AGTGGGCCCGATGAAGAGGCTCTGCTGCAGATGAGGTCGAACATGATTGATGATTC
GTCCGCTCGGATCAGGAGGAACGCCAAGGGGAACTACTGTAAGAAGACCCCACTA
TACATCGACTTCAAGGAGATTGGGTGGGACTCCTGGATCATCGCTCCTCCTGGGTA
TGAAGCCTATGAGTGCCGGGGTGTGTGTAACTACCCTCTGGCGGAGCACCTCACAC
CTACAAAACACGCAATTATTCAGGCCTTGGTCCACCTCAAGAATTCCCAGAAAGCT
TCCAAAGCCTGCTGTGTGCCCACGAAGCTGGATCCCATCTCCATCCTCTATTTAGAT
AAAGGTGTCGTCACCTACAAGTTTAAATATGAAGGGATGGCTGTGTCTGAGTGTGG
CTGTAGATAGGAGAGGAGAGGCGTCCCATG
SEQ ID NO 9: Region 2 of mRNA
CCAUGAAGAGGUCGUCAUGGCUGAACUGCGGUUGUACACGCUGGUGCAGAGAGA
UCGCAUGAUGUAUGAUGGCGUGGACCGUAAAAUUACCAUUUUUGAGGUACUAG
AGAGUGCAGACGGUAGCGAGGAGGAGAGGAGCA
SEQ ID NO 10: Site M2 + M3 of Bmp10 mRNA
GUACAC
SEQ ID NO 11: Site M2 of mRNA
GUA
SEQ ID NO 12: Generic recombinant mRNA sequence
5′-AUUGUCAA*CAGCAGC-3′, A*: m⁶A
List of primers used in the study.
SEQ ID NO: 13 Bmp10 R1-F: CTTCACGGAGCAAGATGGTA;
SEQ ID NO: 14 Bmp10 R1-R: TGTACTCTGGTGGATCCACT
SEQ ID NO: 15 Bmp10 R2-F: CCATGAAGAGGTCGTCAT;
SEQ ID NO 16 Bmp10 R2-R: TGCTCCTCTCCTCCTCGCT
SEQ ID NO 17 Bmp10 R3-F: CTGATGACCAAAGCAATGAC;
SEQ ID NO 18 Bmp10 R3-R: AGAGCCTCTTCATCGGGCCCA
SEQ ID NO 19 Bmp10 R4-F: ATGATTCGTCCGCTCGGATCA;
SEQ ID NO 20: Bmp10 R4-R: CGGCACTCATAGGCTTCATA
SEQ ID NO 21 Bmp10 q-F: CTCAAGACGCTGAACTTGTCG;
SEQ ID NO 22 Bmp10 q-R: GAGAGGATATTTCCGGAGCCC
SEQ ID NO 23 Bmp10 Region2 79 nt primer F: ATGAAGAGGTCGTCATGGCT;
SEQ ID NO 24: Bmp10 Region2 79 nt primer R: CGGTCCACGCCATCATACAT.
SEQ ID NO 25: Biotin labelled RNA Control probe: Biotin-
5′UCGUCAUGGCUGAACUGCGGUUGUACACGCUGGUGCAGAGAGAUCGCAU-3′
SEQ ID NO 26: Biotin-labelled RNA probe with m⁶A: Biotin-
5′UCGUCAUGGCUGAACUGCGGUUGU(m⁶A)CACGCUGGUGCAGAGAGAUCGCAU-3′
SEQ ID NO 27: MBP (amino acids)
MSYYHHHHHHDYDIPTTENLYFQGAMGIRNSKAYVD

1. A method of promoting cardiomyocyte cell proliferation comprising:
providing a demethylase to the cardiomyocyte, wherein the demethylase has enzymatic activity that removes a methyl group from Region 2 of the Bmp10 mRNA.

2. The method of claim 1, wherein the demethylase comprises an amino acid sequence having at least 95% sequence identity to a sequence selected from the group consisting of SEQ ID NO 3, SEQ ID NO: 4 and SEQ ID NO: 5.

3. (canceled)

4. (canceled)

5. The method of claim 1, wherein the demethylase removes a methyl group from site M2+M3 of Region 2.

6. The method of claim 5, wherein site M2+M3 comprises the nucleic acid sequence having at least 95% sequence identity to SEQ ID NO 10.

7. The method of claim 6, wherein site M2 comprises SEQ ID NO 11.

8. The method of claim 1, wherein
i) the demethylase comprises an amino acid sequence having 95% sequence identity to SEQ ID NO 3 and Region 2 comprises a nucleic acid sequence having 95% sequence identity to SEQ ID NO 10; or
ii) the demethylase comprises an amino acid sequence having 95% sequence identity to SEQ ID NO 4 and Region 2 comprises a nucleic acid sequence having 95% sequence identity to SEQ ID NO 10; or
iii) the demethylase comprises an amino acid sequence having 95% sequence identity to SEQ ID NO 5 and Region 2 comprises a nucleic acid sequence having 95% sequence identity to SEQ ID NO 10.

9. (canceled)

10. (canceled)

11. (canceled)

12. (canceled)

13. A method of inducing proliferation in a cardiomyocyte cell comprising:
providing an m⁶A methylation inhibitor to the cardiomyocyte cell; and/or
providing an engineered protein comprising a demethylase to the cell, wherein the demethylase enhances the Brg1 demethylation activity, wherein the engineered protein comprises an amino acid sequence having at least 95% sequence identity to SEQ ID NO 2, wherein the Brg1 demethylation activity is enhanced relative to the base line level of Brg1.

14. The method of claim 13, wherein said method comprises providing 3-DZA to the cardiomyocyte cell.

15. The method of claim 13, wherein the engineered protein comprises an amino acid sequence having 95% sequence identity to a sequence selected from the group consisting of SEQ ID NO: 3, SEQ ID NO 4, and SEQ ID NO: 5.

16. The method of claim 13, wherein said method comprises providing 3-DZA and a demethylase to the cardiomyocyte cell, wherein the demethylase comprises an amino acid sequence having 95% sequence identity to a sequence selected from the group consisting of SEQ ID NO: 3, SEQ ID NO 4, and SEQ ID NO 5.

17. (canceled)

18. A method of enhancing proliferation of cardiomyocytes in accordance with claim 13, said method, comprising:
contacting the cardiomyocytes with an effective amount of a pharmaceutical composition, wherein the pharmaceutical composition includes a demethylase, said demethylase having enzymatic activity that removes a methyl group from Region 2 of Bmp10 mRNA, and a pharmaceutically acceptable carrier.

19. The method of claim 18, wherein the demethylase comprises an amino acid sequence having at least 95% sequence identity to SEQ ID NO 4.

20. The method of claim 18, wherein the demethylase comprises an amino acid sequence having at least 95% sequence identity to SEQ ID NO 5.

21. The method of claim 18, wherein the demethylase is designed to remove a methyl group from site M2+M3 of Region 2.

22. The method of claim 18, wherein site M2+M3 comprises the nucleic acid sequence having at least 95% sequence identity to SEQ ID NO 10.

23. The method of claim 22, wherein site M2+M3 consists essentially of SEQ ID NO 11.

24. The method of claim 18, wherein
i) the demethylase comprises an amino acid sequence having 95% sequence identity to SEQ ID NO 3 and Region 2 comprises a nucleic acid sequence having 95% sequence identity to SEQ ID NO 10; or
ii) the demethylase comprises an amino acid sequence having 95% sequence identity to SEQ ID NO 4 and Region 2 comprises a nucleic acid sequence having 95% sequence identity to SEQ ID NO 10; or
iii) the demethylase comprises an amino acid sequence having 95% sequence identity to SEQ ID NO 5 and Region 2 comprises a nucleic acid sequence having 95% sequence identity to SEQ ID NO 10.

25. (canceled)

26. (canceled)

27. (canceled)

28. (canceled)

29. A culture medium, comprising:
a buffer, and
a pharmaceutical composition that includes a demethylase and a pharmaceutically acceptable carrier, wherein the amount of demethylase present in the culture medium is effective to remove a methyl group from Region 2 of Bmp10 mRNA when in contact with a cardiomyocyte.

30. (canceled)

31. (canceled)

32. The culture media of claim 29 wherein the demethylase comprises an amino acid sequence having at least 95% sequence identity to a sequence selected from the group consisting of SEQ ID NO 3, SEQ ID NO: 4 and SEQ ID NO: 5.


SEQUENCE LISTING (27 sequences)

SEQ ID NO: 1 - protein, 1617 aa, Mus musculus
MSTPDPPLGGTPRPGPSPGPGPSPGAMLGPSPGPSPGSAHSMMGPSPGPPSAGHPMPTQGPGGYPQDNMHQMHKPMESMHEKGMPDDPRYNQMKGMGMRSGAHTGMAPPPSPMDQHSQGYPSPLGGSEHASSPVPASGPSSGPQMSSGPGGAPLDGSDPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPLVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFREYHRSVTGKLQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQPAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKTLKAIEEGTLEEIEEEVRQKKSSRKRKRDSEAGSSTPTTSTRSRDKDEESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 2 - protein, 431 aa, Mus musculus
NGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRL

SEQ ID NO: 3 - protein, 229 aa, Mus musculus
IRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRI

SEQ ID NO: 4 - protein, 17 aa, Mus musculus
IRRLHKVLRPFLLRRLK

SEQ ID NO: 5 - protein, 17 aa, Mus musculus
DWNPHQDLQAQDRAHRI

SEQ ID NO: 6 - protein, 141 aa, Mus musculus
NGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPL

SEQ ID NO: 7 - protein, 109 aa, Mus musculus
NHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRL

SEQ ID NO: 8 - DNA, 1317 bp, Mus musculus
ctaggttggcctgggagctgagcagagagtcatggggtctctggttctgccgctgagcgccgtcttctgcctggtggctcactcggcttctggcagccccattatgggccttgagcagtcgcccctggaagaagacatgcccttctttgatgatatcttcacggagcaagatggtattgacttcaacacactgctgcagagcatgaagaacgagtttctcaagacgctgaacttgtcggacattcctgtgcaggacacgggcagagtggatccaccagagtacatgctggagctctacaacaaattcgccacagaccggacctccatgccgtctgctaacatcatccggagcttcaagaacgaagatctgttttctcaaccagtcacttttaatgggctccggaaatatcctctcctcttcaatgtgtctatccctcaccatgaagaggtcgtcatggctgaactgcggttgtacacgctggtgcagagagatcgcatgatgtatgatggcgtggaccgtaaaattaccatttttgaggtactagagagtgcagacggtagcgaggaggagaggagcatgctggtcttagtatcaacagagatctacggaaccaacagtgagtgggagacatttgacgtcacagatgccaccagacgttggcaaaagtcaggcccatcaacccatcagctggagatccacatagaaagcagacaaaaccaagctgaggacaccggaaggggacaactggaaatagatatgagtgcccaaaataagcatgaccctttgctggttgtgttttctgatgaccaaagcaatgacaaggagcagaaagaagaactgaacgaattgatcacccatgagcaggatctggacctggactcagatgctttcttcagtgggcccgatgaagaggctctgctgcagatgaggtcgaacatgattgatgattcgtccgctcggatcaggaggaacgccaaggggaactactgtaagaagaccccactatacatcgacttcaaggagattgggtgggactcctggatcatcgctcctcctgggtatgaagcctatgagtgccggggtgtgtgtaactaccctctggcggagcacctcacacctacaaaacacgcaattattcaggccttggtccacctcaagaattcccagaaagcttccaaagcctgctgtgtgcccacgaagctggatcccatctccatcctctatttagataaaggtgtcgtcacctacaagtttaaatatgaagggatggctgtgtctgagtgtggctgtagataggagaggagaggcgtcccatg

SEQ ID NO: 9 - RNA, 140 nt, Mus musculus
ccaugaagaggucgucauggcugaacugcgguuguacacgcuggugcagagagaucgcaugauguaugauggcguggaccguaaaauuaccauuuuugagguacuagagagugcagacgguagcgaggaggagaggagca

SEQ ID NO: 10 - RNA, 6 nt, Mus musculus
guacac

SEQ ID NO: 11 - RNA, 3 nt, Mus musculus
gua

SEQ ID NO: 12 - RNA, 15 nt, Mus musculus
  modified_base (8)..(8): Amino acid at position 8 is m6A
auugucaacagcagc

SEQ ID NO: 13 - DNA, 20 bp, Mus musculus
cttcacggagcaagatggta

SEQ ID NO: 14 - DNA, 20 bp, Mus musculus
tgtactctggtggatccact

SEQ ID NO: 15 - DNA, 18 bp, Mus musculus
ccatgaagaggtcgtcat

SEQ ID NO: 16 - DNA, 19 bp, Mus musculus
tgctcctctcctcctcgct

SEQ ID NO: 17 - DNA, 20 bp, Mus musculus
ctgatgaccaaagcaatgac

SEQ ID NO: 18 - DNA, 21 bp, Mus musculus
agagcctcttcatcgggccca

SEQ ID NO: 19 - DNA, 21 bp, Mus musculus
atgattcgtccgctcggatca

SEQ ID NO: 20 - DNA, 20 bp, Mus musculus
cggcactcataggcttcata

SEQ ID NO: 21 - DNA, 21 bp, Mus musculus
ctcaagacgctgaacttgtcg

SEQ ID NO: 22 - DNA, 21 bp, Mus musculus
gagaggatatttccggagccc

SEQ ID NO: 23 - DNA, 20 bp, Mus musculus
atgaagaggtcgtcatggct

SEQ ID NO: 24 - DNA, 20 bp, Mus musculus
cggtccacgccatcatacat

SEQ ID NO: 25 - RNA, 49 nt, Mus musculus
ucgucauggcugaacugcgguuguacacgcuggugcagagagaucgcau

SEQ ID NO: 26 - RNA, 49 nt, Mus musculus; identical to SEQ ID NO: 25
  modified_base (25)..(25): Amino acid at position 25 is m6A

SEQ ID NO: 27 - protein, 36 aa, Mus musculus
MSYYHHHHHHDYDIPTTENLYFQGAMGIRNSKAYVD